SEQUENCE LISTING (92 sequences)

SEQ ID NO: 1 - protein, 277 aa, Homo sapiens
  SIGNAL 1..20
  PEPTIDE 21..277
  DOMAIN 21..193: note = extracellular domain
  DOMAIN 194..215: note = transmembrane domain
  DOMAIN 216..277: note = intracellular domain
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 448 aa, synthetic construct
  REGION 1..448: note = chimeric antibody with mouse variable domain, human constantdomain
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
EVQLQQSGPELEKPGASVKMSCKASGYSFTGYNMNWVKQSNGKSLEWIGNIDPYYGNTNYNQKFKGKATLTVDKSSSTAYLQLKSLTSEDSAVYYCARLGLQLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 219 aa, synthetic construct
  REGION 1..219: note = chimeric antibody with mouse variable domain and human constantdomain
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DVVMTQTPLSLPVSLGDQASISCRSSQSLVHSNGNTYLHWYLQKPGQSPNLLIYKLTNRFFGVPDRFSGSGSGTDFTLKISRVEAEDLGVYFCSQSIHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQTPLSLPVTPGEPASISCRSSQSLVHSNGNTYLHWYLQKPGQSPQLLIYKLTNRFFGVPDRFSGSGSGTDFTLKISRVEAEDVGVYYCSQSIHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 448 aa, synthetic construct; identical to SEQ ID NO: 5
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3

SEQ ID NO: 8 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQTPLSLPVTPGEPASISCRSSQSLVHSNANTYLHWYLQKPGQSPQLLIYKLTNRFFGVPDRFSGSGSGTDFTLKISRVEAEDVGVYYCSQSIHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 219 aa, synthetic construct; identical to SEQ ID NO: 6
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3

SEQ ID NO: 12 - protein, 448 aa, synthetic construct; identical to SEQ ID NO: 10
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3

SEQ ID NO: 13 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 448 aa, synthetic construct
  REGION 1..448: note = humanized antibody with mouse CDRs and human framework andconstant regions
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEEGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 448 aa, synthetic construct
  REGION 1..448: note = humanized antibody with mouse CDRs and human framework andconstant regions
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEEGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 446 aa, synthetic construct
  REGION 1..446: note = chimeric antibody with mouse variable domain and human constantdomain
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLQQSAAELARPGASVKMSCKASGYTFTDLSMHWIKQRPGQGLEWIGYITPSSGYTAYNQKFKGKTTLTADKSSSTAYMQLSSLTSEDSAVYYCARLILQRGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 218 aa, synthetic construct
  REGION 1..218: note = chimeric antibody with mouse variable domain and human constantdomain
  DOMAIN 1..111: note = variable domain
  REGION 24..38: note = MISC_FEATURE - CDRL1
  REGION 54..60: note = MISC_FEATURE - CDRL2
  REGION 93..101: note = MISC_FEATURE - CDRL3
DIVLTQSPASLAVSLGQRATISCRASKNVDSYGNSFMHWYQQKPGQPPKLLIYRASNLESGIPARFGGSGSGTDFTLTINPVEADDVATYYCQQSNEDPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWIKQRPGQGLEWIGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 218 aa, synthetic construct
  REGION 1..218: note = humanized antibody with mouse CDRs and human framework andconstant regions
  DOMAIN 1..111: note = variable domain
  REGION 24..38: note = MISC_FEATURE - CDRL1
  REGION 54..60: note = MISC_FEATURE - CDRL2
  REGION 93..101: note = MISC_FEATURE - CDRL3
DIVMTQSPDSLAVSLGERATINCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 446 aa, synthetic construct; identical to SEQ ID NO: 25
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3

SEQ ID NO: 28 - protein, 218 aa, synthetic construct
  REGION 1..218: note = humanized antibody with mouse CDRs and human framework andconstant regions
  DOMAIN 1..111: note = variable domain
  REGION 24..38: note = MISC_FEATURE - CDRL1
  REGION 54..60: note = MISC_FEATURE - CDRL2
  REGION 93..101: note = MISC_FEATURE - CDRL3
DIVLTQSPDSLAVSLGERATISCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 218 aa, synthetic construct
  REGION 1..218: note = humanized antibody with mouse CDRs and human framework andconstant regions
  DOMAIN 1..111: note = variable domain
  REGION 24..38: note = MISC_FEATURE - CDRL1
  REGION 54..60: note = MISC_FEATURE - CDRL2
  REGION 93..101: note = MISC_FEATURE - CDRL3
DIVLTQSPDSLAVSLGERATINCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 446 aa, synthetic construct
  REGION 1..446: note = humanized antibody with mouse CDRs and human framework andconstant regions
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEEGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 446 aa, synthetic construct
  REGION 1..446: note = humanized antibody with mouse CDRs and human framework andconstant regions
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 446 aa, synthetic construct
  REGION 1..446: note = humanized antibody with mouse CDRs and human framework andconstant regions
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEEGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 451 aa, synthetic construct
  REGION 1..451: note = chimeric antibody with mouse variable domain and human constantdomain
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3
QVQFQQSGAELARPGASVKLSCKASGYTFTSYWMQWVKQRPGQGLEWIGTIYPGDGDSRYNQKFKGKALLTADKSSSIAYMQLNSLASEDSAVYFCARFSLYDGYPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 219 aa, synthetic construct
  REGION 1..219: note = chimeric antibody with mouse variable domain and human constantdomain
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DVVMTQTPLSLPVSLGDQASISCRSSQSLVHRNGNTYLHWYLQKPGQSPKLLIYRVSNRFSGVPDRFSGSGSGTDFTLKISRVEAEDLGIYFCSQSTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 451 aa, synthetic construct
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3
QVQFVQSGAEVKKPGSSVKVSCKASGYTFTSYWMQWVRQAPGQGLEWMGTIYPGDGDSRYNQKFKGKALLTADKSTSTAYMELSSLRSEDTAVYYCARFSLYDGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQSPLSLPVTPGEPASISCRSSQSLVHRNGNTYLHWYLQKPGQSPQLLIYRVSNRFSGVPDRFSGSGSGTDFTLKISRVEAEDVGVYYCSQSTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 451 aa, synthetic construct
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3
QVQFVQSGAEVKKPGSSVKVSCKASGYTFTSYWMQWVRQAPGQGLEWMGTIYPGDGDSRYNQKFKGKALLTADKSTSTAYMELSSLRSEDTAVYYCARFSLYEGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 219 aa, synthetic construct; identical to SEQ ID NO: 43
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3

SEQ ID NO: 46 - protein, 451 aa, synthetic construct; identical to SEQ ID NO: 44
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3

SEQ ID NO: 47 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQSPLSLPVTPGEPASISCRSSQSLVHRNANTYLHWYLQKPGQSPQLLIYRVSNRFSGVPDRFSGSGSGTDFTLKISRVEAEDVGVYYCSQSTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 451 aa, synthetic construct
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3
QVQFVQSGAEVKKPGSSVKVSCKASGYTFTSYWMQWVRQAPGQGLEWMGTIYPGDSDSRYNQKFKGKALLTADKSTSTAYMELSSLRSEDTAVYYCARFSLYEGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 219 aa, synthetic construct; identical to SEQ ID NO: 43
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3

SEQ ID NO: 50 - protein, 451 aa, synthetic construct; identical to SEQ ID NO: 48
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3

SEQ ID NO: 51 - protein, 219 aa, synthetic construct; identical to SEQ ID NO: 47
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3

SEQ ID NO: 52 - protein, 442 aa, synthetic construct
  REGION 1..442: note = chimeric antibody with mouse variable domain and human constantdomain
  DOMAIN 1..113: note = variable domain
  REGION 31..35: note = MISC_FEATURE - CDRH1
  REGION 50..66: note = MISC_FEATURE - CDRH2
  REGION 99..102: note = MISC_FEATURE - CDRH3
EVLLQQSGPELVKPGASVKIPCKASGYKFTDYNMDWVKQSHGKSLEWIGDINPKNGGTIYNLKFKGKATLTVDMSSSTAYMELRSLTSEDTAVYYCVRRMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 214 aa, synthetic construct
  REGION 1..214: note = chimeric antibody with mouse variable domain and human constantdomain
  DOMAIN 1..107: note = variable domain
  REGION 24..34: note = MISC_FEATURE - CDRL1
  REGION 50..56: note = MISC_FEATURE - CDRL2
  REGION 89..97: note = MISC_FEATURE - CDRL3
DIQMTQTTSSLSASLGDRVTISCRASQDISNYLNWYQQKPDGTVKLLIYYISRLHSGVPSRFRGSGSGTDFSLTISNLEQEDIATYFCQQGNTLPYTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 442 aa, synthetic construct
  REGION 1..442: note = humanized antibody with mouse CDRs and human framework andconstant regions
  DOMAIN 1..113: note = variable domain
  REGION 31..35: note = MISC_FEATURE - CDRH1
  REGION 50..66: note = MISC_FEATURE - CDRH2
  REGION 99..102: note = MISC_FEATURE - CDRH3
QVQLVQSGAEVKKPGASVKVSCKASGYTFTDYNMDWVRQAPGQGLEWMGDINPKNGGTIYNLKFKGRVTLTTDTSTSTAYMELRSLRSDDTAVYYCVRRMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 214 aa, synthetic construct
  REGION 1..214: note = humanized antibody with mouse CDRs and human framework andconstant regions
  DOMAIN 1..107: note = variable domain
  REGION 24..34: note = MISC_FEATURE - CDRL1
  REGION 50..56: note = MISC_FEATURE - CDRL2
  REGION 89..97: note = MISC_FEATURE - CDRL3
DIQMTQSPSSLSASVGDRVTITCRASQDISNYLNWYQQKPGKVPKLLIYYISRLHSGVPSRFSGSGSGTDFTLTISSLQPEDVATYYCQQGNTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 442 aa, synthetic construct
  REGION 1..442: note = humanized antibody with mouse CDRs and human framework andconstant regions
  DOMAIN 1..113: note = variable domain
  REGION 31..35: note = MISC_FEATURE - CDRH1
  REGION 50..66: note = MISC_FEATURE - CDRH2
  REGION 99..102: note = MISC_FEATURE - CDRH3
QVQLVQSGAEVKKPGASVKVSCKASGYTFTDYNMDWVRQAPGQGLEWMGDINPKNAGTIYNLKFKGRVTLTVDTSTSTAYMELRSLRSDDTAVYYCVRRMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 214 aa, synthetic construct; identical to SEQ ID NO: 55
  REGION 1..214: note = humanized antibody with mouse CDRs and human framework andconstant regions
  DOMAIN 1..107: note = variable domain
  REGION 24..34: note = MISC_FEATURE - CDRL1
  REGION 50..56: note = MISC_FEATURE - CDRL2
  REGION 89..97: note = MISC_FEATURE - CDRL3

SEQ ID NO: 58 - protein, 441 aa, synthetic construct
  REGION 1..441: note = chimeric antibody with mouse variable domain and human constantdomain
  DOMAIN 1..112: note = variable domain
  REGION 31..35: note = MISC_FEATURE - CDRH1
  REGION 50..66: note = MISC_FEATURE - CDRH2
  REGION 99..101: note = MISC_FEATURE - CDRH3
EVQLVESGGGLVKPGGSLKLSCAASGFTFSSYAMSWVRQSPEKRLEWVAEISGGGSYTYYPDTVTGRFTISRDNAKNTLYLEMSSLRSEDTAMYYCASRAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 219 aa, synthetic construct
  REGION 1..219: note = chimeric antibody with mouse variable domain and human constantdomain
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DVLMTQTPLSLPVSLGDQASISCRSSQSIVHSNGNTYFEWYLQKPGQSPKLLIYKVSNRFSGVPDRFSGSGSGTDFTLKISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 441 aa, synthetic construct
  REGION 1..441: note = humanized antibody with mouse CDRs and human framework andconstant regions
  DOMAIN 1..112: note = variable domain
  REGION 31..35: note = MISC_FEATURE - CDRH1
  REGION 50..66: note = MISC_FEATURE - CDRH2
  REGION 99..101: note = MISC_FEATURE - CDRH3
EVQLVESGGGLVKPGGSLRLSCAASGFTFSSYAMSWVRQAPGKGLEWVSEISGGGSYTYYPDTVTGRFTISRDNAKNSLYLQMNSLRAEDTAVYYCASRAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 441 aa, synthetic construct
  REGION 1..441: note = humanized antibody with mouse CDRs and human framework andconstant regions
  DOMAIN 1..112: note = variable domain
  REGION 31..35: note = MISC_FEATURE - CDRH1
  REGION 50..66: note = MISC_FEATURE - CDRH2
  REGION 99..101: note = MISC_FEATURE - CDRH3
EVQLVESGGGLVKPGGSLRLSCAASGFTFSSYAMSWVRQAPGKGLEWVSEISGGGSYTYYPDTVTGRFTISRDNAKNSLYLQMNSLRAEDTAVYYCASRAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQTPLSLPVTPGEPASISCRSSQSIVHSNGNTYFEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 441 aa, synthetic construct; identical to SEQ ID NO: 60
  REGION 1..441: note = humanized antibody with mouse CDRs and human framework andconstant regions
  DOMAIN 1..112: note = variable domain
  REGION 31..35: note = MISC_FEATURE - CDRH1
  REGION 50..66: note = MISC_FEATURE - CDRH2
  REGION 99..101: note = MISC_FEATURE - CDRH3

SEQ ID NO: 64 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQTPLSLPVTPGEPASISCRSSQSIVHSQGNTYFEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEEGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPDLLGGDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEEGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 107 aa, Homo sapiens [sequence omitted]

SEQ ID NO: 78 - protein, 17 aa, Homo sapiens
MRAWIFFLLCLAGRALA

SEQ ID NO: 79 - protein, 456 aa, synthetic construct
  REGION 1..456: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSATTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 214 aa, synthetic construct
  REGION 1..214: note = Synthetic
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 456 aa, synthetic construct
  REGION 1..456: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSATTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 456 aa, synthetic construct
  REGION 1..456: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSATTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 456 aa, synthetic construct
  REGION 1..456: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSATTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 456 aa, synthetic construct
  REGION 1..456: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSATTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 456 aa, synthetic construct
  REGION 1..456: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSATTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 452 aa, synthetic construct
  REGION 1..452: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 87 - protein, 452 aa, synthetic construct
  REGION 1..452: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 88 - protein, 452 aa, synthetic construct
  REGION 1..452: note = Synthetic
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 89 - protein, 115 aa, Mus musculus
MNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 90 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 91 - protein, 162 aa, Rattus norvegicus
MKILKPYMRNTSILYYLCFLLNSHFLTEAGIHVFILGCVSVGLPKTEANWIDVRYDLEKIESLIQFIHIDTTLYTDSDFHPSCKVTAMNCFLLELQVILHEYSNMTLNETVRNVLYLANSTLSSNKNVIESGCKECEELEERNFTEFLQSFIHIVQMFINTS

SEQ ID NO: 92 - protein, 162 aa, Macaca mulatta
MRISKPHLRSVSIQCYLCLLLNSHFLTEAGIHVFILGCFSAGLPKTEANWVNVISDLKKIEDLIQSMHIDATLYTESDVHPSCKVTAMKCFLLELQVISHESGDTDIHDTVENLIILANNILSSNGNITESGCKECEELEEKNIKEFLQSFVHIVQMFINTS

(19) United States
(12) Patent Application Publication
(10) Pub. No.: US 2024/0400706 A1
(43) Pub. Date: Dec. 5, 2024

(54) COMBINATION OF ANTI-CD40 ANTIBODY AND IL-15 FOR TREATING CANCER

(71) Applicant: The Rockefeller University, New York, NY (US)

(72) Inventors: Jeffrey V. Ravetch, New York, NY (US); Jeffrey Wong, New York, NY (US); David Knorr, New York, NY (US)

(21) Appl. No.: 18/701,493

(22) PCT Filed: Oct. 13, 2022
(86) PCT No.: PCT/US22/78078

§ 371 Date: Apr. 15, 2024

Related U.S. Application Data
(60) Provisional application No. 63/256,153, filed on Oct. 15, 2021.

(51) Int. Cl. C07K 16/28; A61K 38/20; A61K 39/00; A61P 35/00

(52) U.S. Cl. CPC C07K 16/2878; A61K 38/2086; A61P 35/00; A61K 2039/505; C07K 2317/52; C07K 2317/72; C07K 2317/75

(57) ABSTRACT

This disclosure is based, at least in part, on an unexpected discovery that novel combination therapies of an anti-CD40 antibody or antigen binding fragment thereof and an IL-15 polypeptide exhibit synergistic activity in inhibiting tumor growth than any of the monotherapies of the anti-CD40 antibody or antigen binding fragment thereof and the IL-15 polypeptide. Thus, the combination therapy as disclosed herein represents a surprisingly effective therapy for cancer treatment with a reduced risk of treatment-related toxicity.

### CROSS-REFERENCE TO RELATED APPLICATIONS

This application claims priority to U.S. Provisional Application No. 63/256,153, filed on Oct. 15, 2021, the disclosure of which is hereby incorporated by reference in its entirety.

### FIELD OF THE INVENTION

This disclosure relates generally to combination therapies of an anti-CD40 antibody and an IL-15 polypeptide for treating cancer.

### BACKGROUND OF THE INVENTION

Recent research has revealed that human cancers and chronic infections may be treated with agents that modulate the patient's immune response to malignant or infected cells. See, e.g., Reck & Paz-Ares (2015) Semin. Oncol. 42:402. Agonistic anti-CD40 antibodies, such as CP-870893 and dacetuzumab (SGN-40), have been tried for treating cancer based on the belief that they may enhance such an immune response. See, e.g., Kirkwood et al. (2012) CA Cancer J. Clin. 62:309; Vanderheide & Glennie (2013) Clin. Cancer Res. 19:1035. Recent experiments in mice have revealed that anti-CD40 antibodies with enhanced specificity for the inhibitory Fc receptor FcγRIIb have increased anti-tumor efficacy. See, e.g., WO 2012/087928; Smith, et al. (2012) PNAS 109(16):6181-6; Li & Ravetch (2012) Proc. Nat'l Acad. Sci (USA) 109:10966; Wilson et al. (2011) Cancer Cell 19:101; White et al. (2011) J. Immunol. 187:1754.

The need exists for improved therapies containing anti-CD40 antibodies for cancer treatment in human subjects.

### SUMMARY OF THE INVENTION

This disclosure addresses the need mentioned above in a number of aspects by providing a method for treating a cancer. The method comprises administering to a subject in need thereof a therapeutically effective amount of an interleukin-15 (IL-15) polypeptide in combination with a therapeutically effective amount of an isolated antibody or antigen binding fragment thereof that specifically binds to human CD40.

In some embodiments, the anti-CD40 antibody is an anti-CD40 agonist antibody. In some embodiments, the anti-CD40 agonist antibody is selected from 2141, CP-870,893, ChiLob7/6, 12D6, 5F11, 8E8, 5G7, 19G3, APX005M, ADC-1013, CDX-1140, SEA-CD40, SGN-CD40, ABBV-927, and Fc variants thereof. In some embodiments, the anti-CD40 agonist antibody is selected from 2141-V11, CP-870,893-V11, ChiLob7/6-V11, 12D6-V11, 5F11-V11, 8E8-V11, 5G7-V11, 19G3-V11, APX005M-V11, ADC-1013-V11, CDX-1140-V11, SEA-CD40-V11, SGN-CD40-V11, and ABBV-927-V11.

In some embodiments, the anti-CD40 antibody or antigen binding fragment thereof is administered intratumorally. In some embodiments, the cancer is bladder cancer. In some embodiments, the anti-CD40 antibody or antigen binding fragment thereof is administered intravesically.

In some embodiments, one or more doses of the IL-15 polypeptide are administered to the subject prior to or after administering to the subject one or more doses of the anti-CD40 antibody or antigen binding fragment thereof. In some embodiments, one or more doses of the IL-15 polypeptide are administered to the subject concomitantly with administering to the subject one or more doses of the anti-CD40 antibody or antigen binding fragment thereof.

In some embodiments, the IL-15 polypeptide is administered intravenously, subcutaneously, intraperitoneally, intratumorally, or intravesically.

In some embodiments, the therapeutically effective amount of the anti-CD40 antibody or antigen binding fragment thereof comprises from 0.1 to 20 mg/kg of the subject's body weight. In some embodiments, the therapeutically effective amount of the IL-15 polypeptide comprises from 0.1 to 20 mg/kg of the subject's body weight.

In some embodiments, the anti-CD40 antibody or antigen binding fragment thereof comprises three heavy chain complementarity determining regions (HCDR1, HCDR2, and HCDR3) of a heavy chain having an amino acid sequence selected from the group consisting of SEQ ID NOs:79, 81-88 and three light chain complementarity determining regions (LCDR1, LCDR2, and LCDR3) of a light chain having the amino acid sequence of SEQ ID NO: 80.

In some embodiments, the anti-CD40 antibody or antigen binding fragment thereof comprises a heavy chain variable region (HCVR) of a heavy chain having an amino acid sequence selected from the group consisting of SEQ ID NOs:79, 81-88 and a light chain variable region (LCVR) of a light chain having the amino acid sequence of SEQ ID NO: 80.

In some embodiments, the anti-CD40 antibody or antigen binding fragment thereof comprises an Fc region modified to enhance specificity of binding to FcγRIIb, wherein the Fc region comprises a sequence selected SE (SEQ ID NO: 66), SELF (SEQ ID NO: 67), P238D (SEQ ID NO: 68), V4 (SEQ ID NO: 69), V4 D270E (SEQ ID NO: 70), V7 (SEQ ID NO: 71), V8 (SEQ ID NO: 72), V9 (SEQ ID NO: 73), V9 D270E (SEQ ID NO: 74), V11 (SEQ ID NO: 75), and V12 (SEQ ID NO: 76).

In some embodiments, the anti-CD40 antibody or antigen binding fragment thereof comprises a heavy chain having an amino acid sequence selected from the group consisting of SEQ ID NOs: 79, 81-88 and a light chain having the amino acid sequence of SEQ ID NO: 80.

In some embodiments, the IL-15 polypeptide comprises an IL-15 or a variant thereof. In some embodiments, the IL-15 polypeptide comprises an amino acid sequence having at least 80% sequence identity with an amino acid sequence selected from SEQ ID NOs: 89-92 or comprises an amino acid sequence selected from SEQ ID NOs: 89-92.

In some embodiments, the method further comprises administering the subject an additional therapeutic agent or therapy. In some embodiments, the additional therapeutic agent or therapy is selected from radiation, surgery, a chemotherapeutic agent, a cancer vaccine, a PD-1 inhibitor (e.g., an anti-PD-1 antibody), a PD-L1 inhibitor (e.g., an anti-PD-L1 antibody), a B7-H3 inhibitor, a B7-H4 inhibitor, a lymphocyte activation gene 3 (LAG3) inhibitor, a T cell immunoglobulin and mucin-domain containing-3 (TIM3) inhibitor, a galectin 9 (GAL9) inhibitor, a V-domain immunoglobulin (Ig)-containing suppressor of T-cell activation (VISTA) inhibitor, a Killer-Cell Immunoglobulin-Like Receptor (KIR) inhibitor, a B and T lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, a Bacillus Calmette-Guerin (BCG) therapy, a Bacillus Calmette-Guerin (BCG) vaccine, granulocyte-macrophage colony-stimulating factor (GM-CSF), a cytotoxin, an interleukin 6 receptor (IL-6R) inhibitor, an interleukin 4 receptor (IL-4R) inhibitor, an IL-10 inhibitor, IL-2, IL-7, IL-12, IL-21, an antibody-drug conjugate, an anti-inflammatory drug, and combinations thereof.

In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

In some embodiments, the treatment produces a therapeutic effect selected from one or more of: delay in tumor growth, reduction in tumor cell number, tumor regression, prevention or delay of tumor recurrence, increase in survival, partial response, and complete response.

In some embodiments, the tumor growth is inhibited by at least 50% as compared to an untreated patient. In some embodiments, the tumor growth is inhibited by at least 50% as compared to a patient administered the anti-CD40 antibody or antigen binding fragment thereof or the IL-15 polypeptide as a monotherapy.

In another aspect, this disclosure provides a combination or kit comprising an anti-CD40 antibody or antigen binding fragment thereof and an IL-15 polypeptide for use in a method of treating a cancer. The method comprises: selecting a patient with a cancer; and administering to the patient in need thereof a therapeutically effective amount of anti-CD40 antibody or antigen binding fragment thereof in combination with a therapeutically effective amount of the IL-15 polypeptide, wherein the anti-CD40 antibody is selected from 2141-V11, CP-870,893-V11, ChiLob7/6-V11, 12D6-V11, 5F11-V11, 8E8-V11, 5G7-V11, 19G3-V11, APX005M-V11, ADC-1013-V11, CDX-1140-V11, SEA-CD40-V11, SGN-CD40-V11, and ABBV-927-V11.

In some embodiments, the 2141-V11 comprises a heavy chain having an amino acid sequence selected from the group consisting of SEQ ID NOs:79, 81-88 and a light chain having the amino acid sequence of SEQ ID NO: 80.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1i, and 1C are a set of diagrams showing that the Fc-optimized anti-human CD40 agonist antibody (2141-V11) demonstrates enhanced immune stimulatory activity. FIG. 1A shows that human IgG1 antibodies engage activating FcγRs over inhibitory FcγRs underlying ADCC activity, while engagement of inhibitory FcγRs drives agonistic activity. FIG. 1B shows that enhancement of inhibitory receptor (FcγRIIB) binding through Fc-engineering leads to improved agonist activity. FIG. 1C shows enhanced immune cell activation of 2141-V11 compared to other clinically available CD40 antibodies (measured by expansion of OVA-antigen-specific CD8 T cells in peripheral blood of humanized hCD40/hFcγR mice immunized with DEC-OVA in the presence or absence of indicated anti-CD40 antibody Fc variants). **p<0.01, ***p<0.001.

FIGS. 2A and 2B are a set of diagrams showing that intravesical CD40 agonism with the Fc-optimized 2141-V11 antibody induces IL-15Rα upregulation on dendritic cells in the bladder tumor microenvironment. Humanized hCD40/hFcγR mice were orthotopically implanted with MB49 bladder tumors and treated intravesically with a CD40 antibody (2141-V11), BCG, or control IgG on days 6 and 9 post-engraftment. FIGS. 2A and 2B show representative histograms (FIG. 2A) and quantification (FIG. 2B) across mice of IL-15Rα mean fluorescence intensity on type-1 conventional DCs (cDC1), type-2 conventional DCs (cDC2), and macrophages in tumor-bearing bladders as assessed by flow cytometry at day 12 post-engraftment (n=5 mice per group; bars represent SD). **p<0.01, ***p<0.001, ****p<0.0001.

FIGS. 3A, 3B, and 3C are a set of diagrams showing that combination therapy with Fc-optimized anti-CD40 agonist antibody 2141-V11 and IL-15 enhances primary anti-tumor activity. FIG. 3A shows a schematic of the treatment of humanized hCD40/hFcγR mice bearing orthotopic MB49 bladder tumors with intravesical 2141-V11 (days 3, 6, 9, 12) and/or intraperitoneal IL-15 (days 3-12) or control (control IgG and/or vehicle). FIG. 3B shows representative intravital luciferase imaging at day 14 post-tumor implantation and luminescence quantitation across mice (n=5 mice per group; bars represent SD). FIG. 3C shows survival and representative intravital luciferase imaging of surviving mice at day 85 post-tumor implantation treated as outlined in A. *p<0.05, **p<0.01.

FIGS. 4A, 4B, and 4C are a set of diagrams showing that combination therapy with Fc-optimized anti-CD40 agonist antibody 2141-V11 and IL-15 enhances anti-tumor memory responses. FIG. 4A shows a schematic of subcutaneous rechallenge (ten-fold higher dose of tumor cells in the absence of any additional therapy) of humanized hCD40/hFcγR mice surviving long-term (>90 days after initial orthotopic MB49 bladder tumor implantation) following initial therapy with intravesical 2141-V11 (days 3, 6, 9, 12) and/or intraperitoneal IL-15 (days 3-12). FIG. 4B shows tumor volume was monitored and compared with naïve mice receiving an equivalent MB49 tumor cell implant (n=3-5 mice per group; bars represent SD). FIG. 4C shows representative flow cytometry plots and quantification of CD44^{hi}CD122⁺ CD8 T cells (top) and CD11b⁺KLRG1⁺CD27⁻ NK cells (bottom) in the peripheral blood of the mice above. *p<0.05, **p<0.01, ****p<0.0001.

FIG. 5 shows that combination therapy with the Fc-optimized anti-CD40 agonist antibody 2141-V11 and human recombinant IL-15 enhances primary anti-tumor activity. Humanized hCD40/hFcyR mice bearing orthotopic MB49 bladder tumors were treated with intravesical 2141-V11 and/or intravesical recombinant human IL-15 or control (isotype-matched control antibody and/or vehicle) and followed for survival. *p<0.05.

### DETAILED DESCRIPTION OF THE INVENTION

This disclosure is based, at least in part, on an unexpected discovery that novel combination therapies of an anti-CD40 antibody or antigen binding fragment thereof and an IL-15 polypeptide exhibit synergistic activity in inhibiting tumor growth than any of the monotherapies of the anti-CD40 antibody or antigen binding fragment thereof and the IL-15 polypeptide. Thus, the combination therapy as disclosed herein represents a surprisingly effective therapy for cancer treatment with a reduced risk of treatment-related toxicity.

### A. Combination Therapy of Anti-CD40 Antibody and IL-15

Accordingly, in one aspect, this disclosure provides a method for treating a cancer or inhibiting the growth of a tumor. The method comprises administering to a subject in need thereof a therapeutically effective amount of an IL-15 polypeptide in combination with a therapeutically effective amount of an isolated antibody or antigen binding fragment thereof that specifically binds to human CD40.

In some embodiments, the anti-CD40 antibody or antigen binding fragment thereof is administered intratumorally. In some embodiments, the cancer is bladder cancer, and anti-CD40 antibody or antigen binding fragment thereof is administered intravesically. In some embodiments, the IL-15 polypeptide is administered intravenously, subcutaneously, intraperitoneally, intratumorally, or intravesically.

In some embodiments, one or more doses of the IL-15 polypeptide are administered to the subject prior to or after one or more doses of the anti-CD40 antibody or antigen binding fragment thereof. In some embodiments, one or more doses of the IL-15 polypeptide are administered to the subject concomitantly with one or more doses of the anti-CD40 antibody or antigen binding fragment thereof.

As used herein, the term “subject” may be interchangeably used with the term “patient.” The expression “a subject in need thereof” means a human or non-human mammal that exhibits one or more symptoms or indications of cancer and/or who has been diagnosed with cancer. In some embodiments, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes patients who have received one or more cycles of chemotherapy with toxic side effects. In some embodiments, the expression “a subject in need thereof” includes patients with cancer that has been treated but which has subsequently relapsed or metastasized. For example, patients that may have received treatment with one or more anti-cancer agents leading to tumor regression; however, subsequently have relapsed with cancer resistant to the one or more anti-cancer agents (e.g., chemotherapy-resistant cancer) are treated with the methods of the present disclosure.

As used herein, the terms “treating,” “treat,” or the like mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for radiation or surgery, and/or to increase duration of survival of the subject.

In many embodiments, the terms “tumor,” “lesion,” “tumor lesion,” “cancer,” and “malignancy” are used interchangeably and refer to one or more cancerous growths. In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

According to some embodiments, the present disclosure includes methods for treating, delaying, or inhibiting the growth of a tumor. In some embodiments, the present disclosure includes methods to promote tumor regression. In some embodiments, the present disclosure includes methods to reduce tumor cell load or to reduce tumor burden. In some embodiments, the present disclosure includes methods to prevent tumor recurrence.

The methods of the present disclosure, according to some embodiments, comprise administering to a subject in need thereof an anti-CD40 antibody or antigen binding fragment thereof before, after or concurrently with administering to the subject an IL-15 polypeptide. In some embodiments, the methods comprise administering to the subject one or more doses of an anti-CD40 antibody or antigen binding fragment thereof before, after or concurrently with administering to the subject one or more doses of an IL-15 polypeptide.

As used herein, the term “in combination with” also includes sequential or concomitant administration of the anti-CD40 antibody or antigen binding fragment thereof and the IL-15 polypeptide. For example, when administered “before” the IL-15 polypeptide, one or more doses of the anti-CD40 antibody or antigen binding fragment thereof may be administered more than about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of one or more doses of the IL-15 polypeptide.

When administered “after” the IL-15 polypeptide, the anti-CD40 antibody or antigen binding fragment thereof may be administered about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes after the administration of the IL-15 polypeptide.

Administration “concurrent” with the IL-15 polypeptide means that the anti-CD40 antibody or antigen binding fragment thereof is administered to the subject in a separate dosage form within less than 10 minutes (before, after, or at the same time) of administration of the IL-15 polypeptide or administered to the subject as a single combined dosage formulation comprising both the anti-CD40 antibody or antigen binding fragment thereof and the IL-15 polypeptide.

In some embodiments, the disclosed methods may further include administering an anti-tumor therapy. Anti-tumor therapies include, but are not limited to, conventional anti-tumor therapies such as chemotherapy, radiation, surgery, or as elsewhere described herein.

In some embodiments, the treatment produces a therapeutic effect selected from one or more of: delay in tumor growth, reduction in tumor cell number, tumor regression, prevention or delay of tumor recurrence, increase in survival, partial response, and complete response. In some embodiments, the tumor growth in the patient is delayed by at least 10 days as compared to tumor growth in an untreated patient. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to an untreated patient. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to a patient administered the IL-15 polypeptide and the anti-CD40 antibody or antigen binding fragment thereof as monotherapy.

a. Anti-CD40 Antibodies

In some embodiments, the anti-CD40 antibody is an anti-CD40 agonist antibody. Examples of the anti-CD40 antibody that can be used in the disclosed combination therapy have desirable properties for use as therapeutic agents in treating diseases such as cancers. These properties include one or more of the ability to bind to human CD40 with high affinity, acceptably low immunogenicity in human subjects, the ability to bind preferentially to FcγRIIb, and the absence of sequence liabilities that might reduce the chemical stability of the antibody. For example, the anti-CD40 antibody may specifically bind to an epitope on human CD40. Other antibodies that bind to the same or closely related epitopes would likely share these desirable properties, and may be discovered doing competition experiments.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” human CD40, as used in the context of the present disclosure, includes antibodies that bind human CD40 or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human CD40 may, however, have cross-reactivity to other antigens, such as CD40 molecules from other (non-human) species.

In some embodiments, the anti-CD40 agonist antibody can be 2141, CP-870,893, or ChiLob7/6. 2141 an anti-CD40 agonist antibody as set forth in U.S. Pat. Nos. 9,676,862, 7,626,012, and 10,894,835, which are hereby incorporated by reference in their entirety. CP-870,893 is a fully human anti-CD40 agonist antibody and anti-CD40 clone 2141 on a human IgG2 isotype. ChiLob7/6 is the LOB 7/4 (or Lob7.4) antibody set forth in US patent publication US 20090074711A1, which is hereby incorporated by reference in its entirety.

Other anti-CD40 antibodies or antigen-binding fragments thereof that can be used in the context of the methods of the present disclosure include, e.g., 12D6, 5F11, 8E8, 5G7, 19G3, and Fc variants thereof as set forth in U.S. Pat. No. 10,479,838, which is hereby incorporated by reference in its entirety.

Additional anti-CD40 antibodies or antigen-binding fragments thereof that can be used in the context of the methods of the present disclosure include APX005M, ADC-1013, CDX-1140, SEA-CD40, SGN-CD40, and ABBV-927.

APX005M is a humanized IgG1κ antibody consisting of a rabbit hybridoma platform and mutational lineage-guided (MLG) humanization method (Filbert, E. L., et al. Cancer Immunol Immunother 70, 1853-1865 (2021)) as set forth in U.S. Pat. No. 9,676,861, which is hereby incorporated by reference in its entirety. A point mutation was introduced in the Fc-domain at position 267 from serine to aspartic acid (S267E mutation). A version of APX005M lacking S267E mutation (APX005), as well as recombinant analogs of anti-CD40 agonistic antibodies CP-870,893, SGN-40, and ADC-1013, as set forth in U.S. Pat. Nos. 9,676,862, 8,303,955, and 7,338,660, respectively, which are hereby incorporated by reference in their entirety. CDX-1140 is a fully human anti-CD40 antibody as set forth in U.S. Pat. No. 10,865,244, which is hereby incorporated by reference in its entirety. SEA-CD40 is a non-fucosylated anti-CD40 antibody as set forth in WO2016069919A1, which is hereby incorporated by reference in its entirety. ABBV-927 (or Giloralimab) is an agonistic anti-CD40 monoclonal antibody as set forth in U.S. Pat. No. 10,174,121, which is hereby incorporated by reference in its entirety.

Some variability in the antibody sequences disclosed herein may be tolerated and still maintain the desirable properties of the antibody. The CDR regions are delineated using the Kabat system (Kabat, E. A., et al. (1991) Sequences of Proteins of Immunological Interest, Fifth Edition, U.S. Department of Health and Human Services, NIH Publication No. 91-3242). Accordingly, anti-CD40 antibodies or antigen-binding fragments thereof that can be used in the context of the methods of the present disclosure include, anti-huCD40 antibodies comprising CDR sequences that are at least 70%, 75%, 80%, 85%, 90%, 95%, 96%, 97%, 98%, or 99% identical to the CDR sequences of the antibodies disclosed herein (e.g., 2141, CP-870,893, ChiLob7/6, 12D6, 5F11, 8E8, 5G7, and 19G3). The present disclosure also provides anti-huCD40 antibodies comprising heavy and/or light chain variable domain sequences that are at least 70%, 75%, 80%, 85%, 90%, 95%, 96%, 97%, 98%, or 99% identical to the heavy and/or light chain variable domain sequences of an antibody comprising a mutant Fc region having one or more mutations corresponding to one or more mutations in an IgG heavy chain selected from SE (SEQ ID NO: 66), SELF (SEQ ID NO: 67), P238D (SEQ ID NO: 68), V4 (SEQ ID NO: 69), V4 D270E (SEQ ID NO: 70), V7 (SEQ ID NO: 71), V8 (SEQ ID NO: 72), V9 (SEQ ID NO: 73), V9 D270E (SEQ ID NO: 74), V11 (SEQ ID NO: 75), V12 (SEQ ID NO: 76), and humanized derivatives thereof.

In some embodiments, the anti-CD40 agonist antibody is selected from 2141-V11, CP-870,893-V11, ChiLob7/6-V11, 12D6-V11, 5F11-V11, 8E8-V11, 5G7-V11, 19G3-V11, APX005M-V11, ADC-1013-V11, CDX-1140-V11, SEA-CD40-V11, SGN-CD40-V11, and ABBV-927-V11.

TABLE 1
Representative Sequences

| SEQ ID | | OTHER |
|---|---|---|
| NO | SEQUENCES | INFORMATION |
| 1 | MVRLPLQCVLWGCLLTAVHPEPPTACREKQYLINSQCCSLCQPG | Human CD40 |
| | QKLVSDCTEFTETECLPCGESEFLDTWNRETHCHQHKYCDPNLG | (NP_001241) |
| | LRVQQKGTSETDTICTCEEGWHCTSEACESCVLHRSCSPGFGVK | |
| | QIATGVSDTICEPCPVGFFSNVSSAFEKCHPWTSCETKDLVVQQA | |
| | GTNKTDVVCGPQDRLRALVVIPIIFGILFAILLVLVFIKKVAKKPT | |
| | NKAPHPKQEPQEINFPDDLPGSNTAAPVQETLHGCQPVTQEDGK | |
| | ESRISVQERQ | |
| 2 | MIETYNQTSPRSAATGLPISMKIFMYLLTVFLITQMIGSALFAVYL | Human CD40L- |
| | HRRLDKIEDERNLHEDFVFMKTIQRCNTGERSLSLLNCEEIKSQF | gp39 |
| | EGFVKDIMLNKEETKKENSFEMQKGDQNPQIAAHVISEASSKTT | (NP_000065.1) |
| | SVLQWAEKGYYTMSNNLVTLENGKQLTVKRQGLYYIYAQVTF | |
| | CSNREASSQAPFIASLCLKSPGRFERILLRAANTHSSAKPCGQQSI | |
| | HLGGVFELQPGASVFVNVTDPSQVSHGTGFTSFGLLKL | |
| 3 | EVQLQQSGPELEKPGASVKMSCKASGYSFTGYNMNWVKQSNG | 12D6 |
| | KSLEWIGNIDPYYGNTNYNQKFKGKATLTVDKSSSTAYLQLKSL | Chimeric |
| | TSEDSAVYYCARLGLQLYAMDYWGQGTSVTVSSASTKGPSVFP | Heavy Chain |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKRV | |
| | EPKSCDKTHTCPPCPAPELLGGPSVFLFPPKPKDTLMISRTPEVTC | |
| | VVVDVSHEDPEVKFNWYVDGVEVHNAKTKPREEQYNSTYRVV | |
| | SVLTVLHQDWLNGKEYKCKVSNKALPAPIEKTISKAKGQPREPQ | |
| | VYTLPPSREEMTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPG | |
| 4 | DVVMTQTPLSLPVSLGDQASISCRSSQSLVHSNGNTYLHWYLQK | 12D6 |
| | PGQSPNLLIYKLTNRFFGVPDRFSGSGSGTDFTLKISRVEAEDLG | Chimeric |
| | VYFCSQSIHVPWTFGGGTKLEIKRTVAAPSVFIFPPSDEQLKSGT | Light Chain |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| 5 | QVQLVQSGAEVKKPGASVKVSCKASGYTFTGYNMNWVRQAPG | 12D6-03 |
| | QGLEWMGNIDPYYGNTNYNQKFKGRVTMTTDTSTSTAYMELR | Heavy Chain |
| | SLRSDDTAVYYCARLGLQLYAMDYWGQGTTVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVDK | |
| | RVEPKSCDKTHTCPPCPAPELLGGPSVFLFPPKPKDTLMISRTPEV | |
| | TCVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPG | |
| 6 | DIVMTQTPLSLPVTPGEPASISCRSSQSLVHSNGNTYLHWYLQKP | 12D6-03 |
| | GQSPQLLIYKLTNRFFGVPDRFSGSGSGTDFTLKISRVEAEDVGV | Light Chain |
| | YYCSQSIHVPWTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 7 | QVQLVQSGAEVKKPGASVKVSCKASGYTFTGYNMNWVRQAPG | 12D6-22 |
| | QGLEWMGNIDPYYGNTNYNQKFKGRVTMTTDTSTSTAYMELR | Heavy Chain |
| | SLRSDDTAVYYCARLGLQLYAMDYWGQGTTVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVDK | |
| | RVEPKSCDKTHTCPPCPAPELLGGPSVFLFPPKPKDTLMISRTPEV | |
| | TCVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPG | |
| 8 | QVQLVQSGAEVKKPGASVKVSCKASGYTFTGYNMNWVRQAPG | 12D6-22 V9 |
| | QGLEWMGNIDPYYGNTNYNQKFKGRVTMTTDTSTSTAYMELR | Heavy Chain |
| | SLRSDDTAVYYCARLGLQLYAMDYWGQGTTVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVDK | |
| | RVEPKSCDKTHTCPPCPAPELLGDDSVFLFPPKPKDTLMISRTPE | |
| | VTCVVVDVSHEDGEVKFNWYVDGVEVHNAKTKPREEQYNSTY | |
| | RVVSVLTVLHQDWLNGKEYKCKVSNKALPRPIEKTISKAKGQP | |
| | REPQVYTLPPSREEMTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPG | |
| 9 | DIVMTQTPLSLPVTPGEPASISCRSSQSLVHSNANTYLHWYLQKP | 12D6-22/ |
| | GQSPQLLIYKLTNRFFGVPDRFSGSGSGTDFTLKISRVEAEDVGV | 12D6-24 |
| | YYCSQSIHVPWTFGGGTKVEIKRTVAAPSVFIFPPSDEQLKSGTA | Light Chain |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 10 | QVQLVQSGAEVKKPGASVKVSCKASGYTFTGYNMNWVRQAPG | 12D6-23 |
| | QGLEWMGNIDPYYGNTNYNQKFKGRVTMTTDTSTSTAYMELR | Heavy Chain |
| | SLRSDDTAVYYCARLGLQLYALDYWGQGTTVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGGPSVFLFPPKPKDTLMISRTPEVT | |
| | CVVVDVSHEDPEVKFNWYVDGVEVHNAKTKPREEQYNSTYRV | |
| | VSVLTVLHQDWLNGKEYKCKVSNKALPAPIEKTISKAKGQPREP | |
| | QVYTLPPSREEMTKNQVSLTCLVKGFYPSDIAVEWESNGQPENN | |
| | YKTTPPVLDSDGSFFLYSKLTVDKSRWQQGNVFSCSVMHEALH | |
| | NHYTQKSLSLSPG | |
| 11 | DIVMTQTPLSLPVTPGEPASISCRSSQSLVHSNGNTYLHWYLQKP | 12D6-23 |
| | GQSPQLLIYKLTNRFFGVPDRFSGSGSGTDFTLKISRVEAEDVGV | Light Chain |
| | YYCSQSIHVPWTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 12 | QVQLVQSGAEVKKPGASVKVSCKASGYTFTGYNMNWVRQAPG | 12D6-24 |
| | QGLEWMGNIDPYYGNTNYNQKFKGRVTMTTDTSTSTAYMELR | Heavy Chain |
| | SLRSDDTAVYYCARLGLQLYALDYWGQGTTVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGGPSVFLFPPKPKDTLMISRTPEVT | |
| | CVVVDVSHEDPEVKFNWYVDGVEVHNAKTKPREEQYNSTYRV | |
| | VSVLTVLHQDWLNGKEYKCKVSNKALPAPIEKTISKAKGQPREP | |
| | QVYTLPPSREEMTKNQVSLTCLVKGFYPSDIAVEWESNGQPENN | |
| | YKTTPPVLDSDGSFFLYSKLTVDKSRWQQGNVFSCSVMHEALH | |
| | NHYTQKSLSLSPG | |
| 13 | QVQLVQSGAEVKKPGASVKVSCKASGYTFTGYNMNWVRQAPG | 12D6-24 |
| | QGLEWMGNIDPYYGNTNYNQKFKGRVTMTTDTSTSTAYMELR | P238D |
| | SLRSDDTAVYYCARLGLQLYALDYWGQGTTVTVSSASTKGPSV | Heavy Chain |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGGDSVFLFPPKPKDTLMISRTPEV | |
| | TCVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPG | |
| 14 | QVQLVQSGAEVKKPGASVKVSCKASGYTFTGYNMNWVRQAPG | 12D6-24 SE |
| | QGLEWMGNIDPYYGNTNYNQKFKGRVTMTTDTSTSTAYMELR | Heavy Chain |
| | SLRSDDTAVYYCARLGLQLYALDYWGQGTTVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGGPSVFLFPPKPKDTLMISRTPEVT | |
| | CVVVDVEHEDPEVKFNWYVDGVEVHNAKTKPREEQYNSTYRV | |
| | VSVLTVLHQDWLNGKEYKCKVSNKALPAPIEKTISKAKGQPREP | |
| | QVYTLPPSREEMTKNQVSLTCLVKGFYPSDIAVEWESNGQPENN | |
| | YKTTPPVLDSDGSFFLYSKLTVDKSRWQQGNVFSCSVMHEALH | |
| | NHYTQKSLSLSPG | |
| 15 | QVQLVQSGAEVKKPGASVKVSCKASGYTFTGYNMNWVRQAPG | 12D6-24 |
| | QGLEWMGNIDPYYGNTNYNQKFKGRVTMTTDTSTSTAYMELR | SELF |
| | SLRSDDTAVYYCARLGLQLYALDYWGQGTTVTVSSASTKGPSV | Heavy Chain |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGGPSVFLFPPKPKDTLMISRTPEVT | |
| | CVVVDVEHEDPEVKFNWYVDGVEVHNAKTKPREEQYNSTYRV | |
| | VSVLTVLHQDWLNGKEYKCKVSNKAFPAPIEKTISKAKGQPREP | |
| | QVYTLPPSREEMTKNQVSLTCLVKGFYPSDIAVEWESNGQPENN | |
| | YKTTPPVLDSDGSFFLYSKLTVDKSRWQQGNVFSCSVMHEALH | |
| | NHYTQKSLSLSPG | |
| 16 | QVQLVQSGAEVKKPGASVKVSCKASGYTFTGYNMNWVRQAPG | 12D6-24 V4 |
| | QGLEWMGNIDPYYGNTNYNQKFKGRVTMTTDTSTSTAYMELR | Heavy Chain |
| | SLRSDDTAVYYCARLGLQLYALDYWGQGTTVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGGDSVFLFPPKPKDTLMISRTPEV | |
| | TCVVVDVSHEDGEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPG | |
| 17 | QVQLVQSGAEVKKPGASVKVSCKASGYTFTGYNMNWVRQAPG | 12D6-24 V4 |
| | QGLEWMGNIDPYYGNTNYNQKFKGRVTMTTDTSTSTAYMELR | D270E |
| | SLRSDDTAVYYCARLGLQLYALDYWGQGTTVTVSSASTKGPSV | Heavy Chain |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGGDSVFLFPPKPKDTLMISRTPEV | |
| | TCVVVDVSHEEGEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPG | |
| 18 | QVQLVQSGAEVKKPGASVKVSCKASGYTFTGYNMNWVRQAPG | 12D6-24 V8 |
| | QGLEWMGNIDPYYGNTNYNQKFKGRVTMTTDTSTSTAYMELR | Heavy Chain |
| | SLRSDDTAVYYCARLGLQLYALDYWGQGTTVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGDDSVFLFPPKPKDTLMISRTPEV | |
| | TCVVVDVSDEDGEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPG | |
| 19 | QVQLVQSGAEVKKPGASVKVSCKASGYTFTGYNMNWVRQAPG | 12D6-24 V9 |
| | QGLEWMGNIDPYYGNTNYNQKFKGRVTMTTDTSTSTAYMELR | Heavy Chain |
| | SLRSDDTAVYYCARLGLQLYALDYWGQGTTVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGDDSVFLFPPKPKDTLMISRTPEV | |
| | TCVVVDVSHEDGEVKFNWYVDGVEVHNAKTKPREEQYNSTYR | |
| | VVSVLTVLHQDWLNGKEYKCKVSNKALPRPIEKTISKAKGQPRE | |
| | PQVYTLPPSREEMTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPG | |
| 20 | QVQLVQSGAEVKKPGASVKVSCKASGYTFTGYNMNWVRQAPG | 12D6-24 V9 |
| | QGLEWMGNIDPYYGNTNYNQKFKGRVTMTTDTSTSTAYMELR | D270E |
| | SLRSDDTAVYYCARLGLQLYALDYWGQGTTVTVSSASTKGPSV | Heavy Chain |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGDDSVFLFPPKPKDTLMISRTPEV | |
| | TCVVVDVSHEEGEVKFNWYVDGVEVHNAKTKPREEQYNSTYR | |
| | VVSVLTVLHQDWLNGKEYKCKVSNKALPRPIEKTISKAKGQPRE | |
| | PQVYTLPPSREEMTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPG | |
| 21 | QVQLVQSGAEVKKPGASVKVSCKASGYTFTGYNMNWVRQAPG | 12D6-24 |
| | QGLEWMGNIDPYYGNTNYNQKFKGRVTMTTDTSTSTAYMELR | V11 Heavy |
| | SLRSDDTAVYYCARLGLQLYALDYWGQGTTVTVSSASTKGPSV | Chain |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPELLGDDSVFLFPPKPKDTLMISRTPEV | |
| | TCVVVDVSDEDGEVKFNWYVDGVEVHNAKTKPREEQYNSTYR | |
| | VVSVLTVLHQDWLNGKEYKCKVSNKALPRPIEKTISKAKGQPRE | |
| | PQVYTLPPSREEMTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPG | |
| 22 | QVQLVQSGAEVKKPGASVKVSCKASGYTFTGYNMNWVRQAPG | 12D6-24 |
| | QGLEWMGNIDPYYGNTNYNQKFKGRVTMTTDTSTSTAYMELR | V12 Heavy |
| | SLRSDDTAVYYCARLGLQLYALDYWGQGTTVTVSSASTKGPSV | Chain |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKR | |
| | VEPKSCDKTHTCPPCPAPDLLGDDSVFLFPPKPKDTLMISRTPEV | |
| | TCVVVDVSDEDGEVKFNWYVDGVEVHNAKTKPREEQYNSTYR | |
| | VVSVLTVLHQDWLNGKEYKCKVSNKALPRPIEKTISKAKGQPRE | |
| | PQVYTLPPSREEMTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPG | |
| 23 | QVQLQQSAAELARPGASVKMSCKASGYTFTDLSMHWIKQRPGQ | 5F11 |
| | GLEWIGYITPSSGYTAYNQKFKGKTTLTADKSSSTAYMQLSSLT | Chimeric |
| | SEDSAVYYCARLILQRGAYWGQGTLVTVSAASTKGPSVFPLAPS | Heavy Chain |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLT | |
| | VLHQDWLNGKEYKCKVSNKALPAPIEKTISKAKGQPREPQVYT | |
| | LPPSREEMTKNQVSLTCLVKGFYPSDIAVEWESNGQPENNYKTT | |
| | PPVLDSDGSFFLYSKLTVDKSRWQQGNVFSCSVMHEALHNHYT | |
| | QKSLSLSPG | |
| 24 | DIVLTQSPASLAVSLGQRATISCRASKNVDSYGNSFMHWYQQKP | 5F11 |
| | GQPPKLLIYRASNLESGIPARFGGSGSGTDFTLTINPVEADDVAT | Chimeric |
| | YYCQQSNEDPLTFGAGTKLELKRTVAAPSVFIFPPSDEQLKSGTA | Light Chain |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 25 | QVQLVQSGAEVKKPGSSVKVSCKASGYTFTDLSMHWIKQRPGQ | 5F11-17 |
| | GLEWIGYITPSSGYTAYNQKFKGKTTLTADKSTSTAYMELSSLR | Heavy Chain |
| | SEDTAVYYCARLILQRGAYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLT | |
| | VLHQDWLNGKEYKCKVSNKALPAPIEKTISKAKGQPREPQVYT | |
| | LPPSREEMTKNQVSLTCLVKGFYPSDIAVEWESNGQPENNYKTT | |
| | PPVLDSDGSFFLYSKLTVDKSRWQQGNVFSCSVMHEALHNHYT | |
| | QKSLSLSPG | |
| 26 | DIVMTQSPDSLAVSLGERATINCRASKNVDSYGNSFMHWYQQK | 5F11-17 |
| | PGQPPKLLIYRASNLESGVPDRFSGSGSGTDFTLTISSLQAEDVAV | Light Chain |
| | YYCQQSNEDPLTFGQGTKLEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 27 | QVQLVQSGAEVKKPGSSVKVSCKASGYTFTDLSMHWIKQRPGQ | 5F11-23 |
| | GLEWIGYITPSSGYTAYNQKFKGKTTLTADKSTSTAYMELSSLR | Heavy Chain |
| | SEDTAVYYCARLILQRGAYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLT | |
| | VLHQDWLNGKEYKCKVSNKALPAPIEKTISKAKGQPREPQVYT | |
| | LPPSREEMTKNQVSLTCLVKGFYPSDIAVEWESNGQPENNYKTT | |
| | PPVLDSDGSFFLYSKLTVDKSRWQQGNVFSCSVMHEALHNHYT | |
| | QKSLSLSPG | |
| 28 | DIVLTQSPDSLAVSLGERATISCRASKNVDSYGNSFMHWYQQKP | 5F11-23 |
| | GQPPKLLIYRASNLESGVPDRFSGSGSGTDFTLTISSLQAEDVAV | Light Chain |
| | YYCQQSNEDPLTFGQGTKLEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 29 | QVQLVQSGAEVKKPGSSVKVSCKASGYTFTDLSMHWVRQAPG | 5F11-45 |
| | QGLEWMGYITPSSGYTAYNQKFKGKTTLTADKSTSTAYMELSS | Heavy Chain |
| | LRSEDTAVYYCARLILQRGAYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGGPSVFLFPPKPKDTLMISRTPEVTCVV | |
| | VDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSREEMTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPG | |
| 30 | DIVLTQSPDSLAVSLGERATINCRASKNVDSYGNSFMHWYQQKP | 5F11-45 |
| | GQPPKLLIYRASNLESGVPDRFSGSGSGTDFTLTISSLQAEDVAV | Light Chain |
| | YYCQQSNEDPLTFGQGTKLEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 31 | QVQLVQSGAEVKKPGSSVKVSCKASGYTFTDLSMHWVRQAPG | 5F11-45 SE |
| | QGLEWMGYITPSSGYTAYNQKFKGKTTLTADKSTSTAYMELSS | Heavy Chain |
| | LRSEDTAVYYCARLILQRGAYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGGPSVFLFPPKPKDTLMISRTPEVTCVV | |
| | VDVEHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSREEMTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPG | |
| 32 | QVQLVQSGAEVKKPGSSVKVSCKASGYTFTDLSMHWVRQAPG | 5F11-45 |
| | QGLEWMGYITPSSGYTAYNQKFKGKTTLTADKSTSTAYMELSS | SELF |
| | LRSEDTAVYYCARLILQRGAYWGQGTLVTVSSASTKGPSVFPLA | Heavy Chain |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGGPSVFLFPPKPKDTLMISRTPEVTCVV | |
| | VDVEHEDPEVKFNWYVDGVEVHNAKTKPREEQYNSTYRVVSV | |
| | LTVLHQDWLNGKEYKCKVSNKAFPAPIEKTISKAKGQPREPQVY | |
| | TLPPSREEMTKNQVSLTCLVKGFYPSDIAVEWESNGQPENNYKT | |
| | TPPVLDSDGSFFLYSKLTVDKSRWQQGNVFSCSVMHEALHNHY | |
| | TQKSLSLSPG | |
| 33 | QVQLVQSGAEVKKPGSSVKVSCKASGYTFTDLSMHWVRQAPG | 5F11-45 V4 |
| | QGLEWMGYITPSSGYTAYNQKFKGKTTLTADKSTSTAYMELSS | Heavy Chain |
| | LRSEDTAVYYCARLILQRGAYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGGDSVFLFPPKPKDTLMISRTPEVTCVV | |
| | VDVSHEDGEVKFNWYVDGVEVHNAKTKPREEQYNSTYRVVSV | |
| | LTVLHQDWLNGKEYKCKVSNKALPAPIEKTISKAKGQPREPQV | |
| | YTLPPSREEMTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPG | |
| 34 | QVQLVQSGAEVKKPGSSVKVSCKASGYTFTDLSMHWVRQAPG | 5F11-45 |
| | QGLEWMGYITPSSGYTAYNQKFKGKTTLTADKSTSTAYMELSS | D270E V4 |
| | LRSEDTAVYYCARLILQRGAYWGQGTLVTVSSASTKGPSVFPLA | Heavy Chain |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGGDSVFLFPPKPKDTLMISRTPEVTCVV | |
| | VDVSHEEGEVKFNWYVDGVEVHNAKTKPREEQYNSTYRVVSV | |
| | LTVLHQDWLNGKEYKCKVSNKALPAPIEKTISKAKGQPREPQV | |
| | YTLPPSREEMTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPG | |
| 35 | QVQLVQSGAEVKKPGSSVKVSCKASGYTFTDLSMHWVRQAPG | 5F11-45 V8 |
| | QGLEWMGYITPSSGYTAYNQKFKGKTTLTADKSTSTAYMELSS | Heavy Chain |
| | LRSEDTAVYYCARLILQRGAYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGDDSVFLFPPKPKDTLMISRTPEVTCVV | |
| | VDVSDEDGEVKFNWYVDGVEVHNAKTKPREEQYNSTYRVVSV | |
| | LTVLHQDWLNGKEYKCKVSNKALPAPIEKTISKAKGQPREPQV | |
| | YTLPPSREEMTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPG | |
| 36 | QVQLVQSGAEVKKPGSSVKVSCKASGYTFTDLSMHWVRQAPG | 5F11-45 V9 |
| | QGLEWMGYITPSSGYTAYNQKFKGKTTLTADKSTSTAYMELSS | Heavy Chain |
| | LRSEDTAVYYCARLILQRGAYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGDDSVFLFPPKPKDTLMISRTPEVTCVV | |
| | VDVSHEDGEVKFNWYVDGVEVHNAKTKPREEQYNSTYRVVSV | |
| | LTVLHQDWLNGKEYKCKVSNKALPRPIEKTISKAKGQPREPQVY | |
| | TLPPSREEMTKNQVSLTCLVKGFYPSDIAVEWESNGQPENNYKT | |
| | TPPVLDSDGSFFLYSKLTVDKSRWQQGNVFSCSVMHEALHNHY | |
| | TQKSLSLSPG | |
| 37 | QVQLVQSGAEVKKPGSSVKVSCKASGYTFTDLSMHWVRQAPG | 5F11-45 V9 |
| | QGLEWMGYITPSSGYTAYNQKFKGKTTLTADKSTSTAYMELSS | D270E |
| | LRSEDTAVYYCARLILQRGAYWGQGTLVTVSSASTKGPSVFPLA | Heavy Chain |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGDDSVFLFPPKPKDTLMISRTPEVTCVV | |
| | VDVSHEEGEVKFNWYVDGVEVHNAKTKPREEQYNSTYRVVSV | |
| | LTVLHQDWLNGKEYKCKVSNKALPRPIEKTISKAKGQPREPQVY | |
| | TLPPSREEMTKNQVSLTCLVKGFYPSDIAVEWESNGQPENNYKT | |
| | TPPVLDSDGSFFLYSKLTVDKSRWQQGNVFSCSVMHEALHNHY | |
| | TQKSLSLSPG | |
| 38 | QVQLVQSGAEVKKPGSSVKVSCKASGYTFTDLSMHWVRQAPG | 5F11-45 |
| | QGLEWMGYITPSSGYTAYNQKFKGKTTLTADKSTSTAYMELSS | V11 Heavy |
| | LRSEDTAVYYCARLILQRGAYWGQGTLVTVSSASTKGPSVFPLA | Chain |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPELLGDDSVFLFPPKPKDTLMISRTPEVTCVV | |
| | VDVSDEDGEVKFNWYVDGVEVHNAKTKPREEQYNSTYRVVSV | |
| | LTVLHQDWLNGKEYKCKVSNKALPRPIEKTISKAKGQPREPQVY | |
| | TLPPSREEMTKNQVSLTCLVKGFYPSDIAVEWESNGQPENNYKT | |
| | TPPVLDSDGSFFLYSKLTVDKSRWQQGNVFSCSVMHEALHNHY | |
| | TQKSLSLSPG | |
| 39 | QVQLVQSGAEVKKPGSSVKVSCKASGYTFTDLSMHWVRQAPG | 5F11-45 |
| | QGLEWMGYITPSSGYTAYNQKFKGKTTLTADKSTSTAYMELSS | V12 Heavy |
| | LRSEDTAVYYCARLILQRGAYWGQGTLVTVSSASTKGPSVFPLA | Chain |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKRVEPK | |
| | SCDKTHTCPPCPAPDLLGDDSVFLFPPKPKDTLMISRTPEVTCVV | |
| | VDVSDEDGEVKFNWYVDGVEVHNAKTKPREEQYNSTYRVVSV | |
| | LTVLHQDWLNGKEYKCKVSNKALPRPIEKTISKAKGQPREPQVY | |
| | TLPPSREEMTKNQVSLTCLVKGFYPSDIAVEWESNGQPENNYKT | |
| | TPPVLDSDGSFFLYSKLTVDKSRWQQGNVFSCSVMHEALHNHY | |
| | TQKSLSLSPG | |
| 40 | QVQFQQSGAELARPGASVKLSCKASGYTFTSYWMQWVKQRPG | 8E8 |
| | QGLEWIGTIYPGDGDSRYNQKFKGKALLTADKSSSIAYMQLNSL | Chimeric |
| | ASEDSAVYFCARFSLYDGYPYYFDYWGQGTTLTVSSASTKGPS | Heavy Chain |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVDK | |
| | RVEPKSCDKTHTCPPCPAPELLGGPSVFLFPPKPKDTLMISRTPEV | |
| | TCVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPG | |
| 41 | DVVMTQTPLSLPVSLGDQASISCRSSQSLVHRNGNTYLHWYLQ | 8E8 |
| | KPGQSPKLLIYRVSNRFSGVPDRFSGSGSGTDFTLKISRVEAEDL | Chimeric |
| | GIYFCSQSTHFPYTFGGGTKLEIKRTVAAPSVFIFPPSDEQLKSGT | Light Chain |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| 42 | QVQFVQSGAEVKKPGSSVKVSCKASGYTFTSYWMQWVRQAPG | 8E8-56 |
| | QGLEWMGTIYPGDGDSRYNQKFKGKALLTADKSTSTAYMELSS | Heavy Chain |
| | LRSEDTAVYYCARFSLYDGYPYYFDYWGQGTTVTVSSASTKGP | |
| | SVFPLAPSSKSTSGGTAALGCLVKDYFPEPVTVSWNSGALTSGV | |
| | HTFPAVLQSSGLYSLSSVVTVPSSSLGTQTYICNVNHKPSNTKVD | |
| | KRVEPKSCDKTHTCPPCPAPELLGGPSVFLFPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSREEMTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPG | |
| 43 | DIVMTQSPLSLPVTPGEPASISCRSSQSLVHRNGNTYLHWYLQKP | 8E8-56 |
| | GQSPQLLIYRVSNRFSGVPDRFSGSGSGTDFTLKISRVEAEDVGV | Light Chain |
| | YYCSQSTHFPYTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 44 | QVQFVQSGAEVKKPGSSVKVSCKASGYTFTSYWMQWVRQAPG | 8E8-62 |
| | QGLEWMGTIYPGDGDSRYNQKFKGKALLTADKSTSTAYMELSS | Heavy Chain |
| | LRSEDTAVYYCARFSLYEGYPYYFDYWGQGTTVTVSSASTKGP | |
| | SVFPLAPSSKSTSGGTAALGCLVKDYFPEPVTVSWNSGALTSGV | |
| | HTFPAVLQSSGLYSLSSVVTVPSSSLGTQTYICNVNHKPSNTKVD | |
| | KRVEPKSCDKTHTCPPCPAPELLGGPSVFLFPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSREEMTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPG | |
| 45 | DIVMTQSPLSLPVTPGEPASISCRSSQSLVHRNGNTYLHWYLQKP | 8E8-62 |
| | GQSPQLLIYRVSNRFSGVPDRFSGSGSGTDFTLKISRVEAEDVGV | Light Chain |
| | YYCSQSTHFPYTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 46 | QVQFVQSGAEVKKPGSSVKVSCKASGYTFTSYWMQWVRQAPG | 8E8-67 |
| | QGLEWMGTIYPGDGDSRYNQKFKGKALLTADKSTSTAYMELSS | Heavy Chain |
| | LRSEDTAVYYCARFSLYEGYPYYFDYWGQGTTVTVSSASTKGP | |
| | SVFPLAPSSKSTSGGTAALGCLVKDYFPEPVTVSWNSGALTSGV | |
| | HTFPAVLQSSGLYSLSSVVTVPSSSLGTQTYICNVNHKPSNTKVD | |
| | KRVEPKSCDKTHTCPPCPAPELLGGPSVFLFPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSREEMTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPG | |
| 47 | DIVMTQSPLSLPVTPGEPASISCRSSQSLVHRNANTYLHWYLQKP | 8E8-67 |
| | GQSPQLLIYRVSNRFSGVPDRFSGSGSGTDFTLKISRVEAEDVGV | Light Chain |
| | YYCSQSTHFPYTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 48 | QVQFVQSGAEVKKPGSSVKVSCKASGYTFTSYWMQWVRQAPG | 8E8-70 |
| | QGLEWMGTIYPGDSDSRYNQKFKGKALLTADKSTSTAYMELSS | Heavy Chain |
| | LRSEDTAVYYCARFSLYEGYPYYFDYWGQGTTVTVSSASTKGP | |
| | SVFPLAPSSKSTSGGTAALGCLVKDYFPEPVTVSWNSGALTSGV | |
| | HTFPAVLQSSGLYSLSSVVTVPSSSLGTQTYICNVNHKPSNTKVD | |
| | KRVEPKSCDKTHTCPPCPAPELLGGPSVFLFPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSREEMTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPG | |
| 49 | DIVMTQSPLSLPVTPGEPASISCRSSQSLVHRNGNTYLHWYLQKP | 8E8-70 |
| | GQSPQLLIYRVSNRFSGVPDRFSGSGSGTDFTLKISRVEAEDVGV | Light Chain |
| | YYCSQSTHFPYTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 50 | QVQFVQSGAEVKKPGSSVKVSCKASGYTFTSYWMQWVRQAPG | 8E8-71 |
| | QGLEWMGTIYPGDSDSRYNQKFKGKALLTADKSTSTAYMELSS | Heavy Chain |
| | LRSEDTAVYYCARFSLYEGYPYYFDYWGQGTTVTVSSASTKGP | |
| | SVFPLAPSSKSTSGGTAALGCLVKDYFPEPVTVSWNSGALTSGV | |
| | HTFPAVLQSSGLYSLSSVVTVPSSSLGTQTYICNVNHKPSNTKVD | |
| | KRVEPKSCDKTHTCPPCPAPELLGGPSVFLFPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSREEMTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPG | |
| 51 | DIVMTQSPLSLPVTPGEPASISCRSSQSLVHRNANTYLHWYLQKP | 8E8-71 |
| | GQSPQLLIYRVSNRFSGVPDRFSGSGSGTDFTLKISRVEAEDVGV | Light Chain |
| | YYCSQSTHFPYTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 52 | EVLLQQSGPELVKPGASVKIPCKASGYKFTDYNMDWVKQSHGK | 5G7 |
| | SLEWIGDINPKNGGTIYNLKFKGKATLTVDMSSSTAYMELRSLT | Chimeric |
| | SEDTAVYYCVRRMDYWGQGTSVTVSSASTKGPSVFPLAPSSKST | Heavy Chain |
| | SGGTAALGCLVKDYFPEPVTVSWNSGALTSGVHTFPAVLQSSGL | |
| | YSLSSVVTVPSSSLGTQTYICNVNHKPSNTKVDKRVEPKSCDKT | |
| | HTCPPCPAPELLGGPSVFLFPPKPKDTLMISRTPEVTCVVVDVSH | |
| | EDPEVKFNWYVDGVEVHNAKTKPREEQYNSTYRVVSVLTVLH | |
| | QDWLNGKEYKCKVSNKALPAPIEKTISKAKGQPREPQVYTLPPS | |
| | REEMTKNQVSLTCLVKGFYPSDIAVEWESNGQPENNYKTTPPVL | |
| | DSDGSFFLYSKLTVDKSRWQQGNVFSCSVMHEALHNHYTQKSL | |
| | SLSPG | |
| 53 | DIQMTQTTSSLSASLGDRVTISCRASQDISNYLNWYQQKPDGTV | 5G7 |
| | KLLIYYISRLHSGVPSRFRGSGSGTDFSLTISNLEQEDIATYFCQQ | Chimeric |
| | GNTLPYTFGGGTNLEIKRTVAAPSVFIFPPSDEQLKSGTASVVCL | Light Chain |
| | LNNFYPREAKVQWKVDNALQSGNSQESVTEQDSKDSTYSLSST | |
| | LTLSKADYEKHKVYACEVTHQGLSSPVTKSFNRGEC | |
| 54 | QVQLVQSGAEVKKPGASVKVSCKASGYTFTDYNMDWVRQAPG | 5G7-22 |
| | QGLEWMGDINPKNGGTIYNLKFKGRVTLTTDTSTSTAYMELRSL | Heavy Chain |
| | RSDDTAVYYCVRRMDYWGQGTTVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKRVEPKSCD | |
| | KTHTCPPCPAPELLGGPSVFLFPPKPKDTLMISRTPEVTCVVVDV | |
| | SHEDPEVKFNWYVDGVEVHNAKTKPREEQYNSTYRVVSVLTVL | |
| | HQDWLNGKEYKCKVSNKALPAPIEKTISKAKGQPREPQVYTLPP | |
| | SREEMTKNQVSLTCLVKGFYPSDIAVEWESNGQPENNYKTTPPV | |
| | LDSDGSFFLYSKLTVDKSRWQQGNVFSCSVMHEALHNHYTQKS | |
| | LSLSPG | |
| 55 | DIQMTQSPSSLSASVGDRVTITCRASQDISNYLNWYQQKPGKVP | 5G7-22 |
| | KLLIYYISRLHSGVPSRFSGSGSGTDFTLTISSLQPEDVATYYCQQ | Light Chain |
| | GNTLPYTFGGGTKVEIKRTVAAPSVFIFPPSDEQLKSGTASVVCL | |
| | LNNFYPREAKVQWKVDNALQSGNSQESVTEQDSKDSTYSLSST | |
| | LTLSKADYEKHKVYACEVTHQGLSSPVTKSFNRGEC | |
| 56 | QVQLVQSGAEVKKPGASVKVSCKASGYTFTDYNMDWVRQAPG | 5G7-25 |
| | QGLEWMGDINPKNAGTIYNLKFKGRVTLTVDTSTSTAYMELRS | Heavy Chain |
| | LRSDDTAVYYCVRRMDYWGQGTTVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKRVEPKSCD | |
| | KTHTCPPCPAPELLGGPSVFLFPPKPKDTLMISRTPEVTCVVVDV | |
| | SHEDPEVKFNWYVDGVEVHNAKTKPREEQYNSTYRVVSVLTVL | |
| | HQDWLNGKEYKCKVSNKALPAPIEKTISKAKGQPREPQVYTLPP | |
| | SREEMTKNQVSLTCLVKGFYPSDIAVEWESNGQPENNYKTTPPV | |
| | LDSDGSFFLYSKLTVDKSRWQQGNVFSCSVMHEALHNHYTQKS | |
| | LSLSPG | |
| 57 | DIQMTQSPSSLSASVGDRVTITCRASQDISNYLNWYQQKPGKVP | 5G7-25 |
| | KLLIYYISRLHSGVPSRFSGSGSGTDFTLTISSLQPEDVATYYCQQ | Light Chain |
| | GNTLPYTFGGGTKVEIKRTVAAPSVFIFPPSDEQLKSGTASVVCL | |
| | LNNFYPREAKVQWKVDNALQSGNSQESVTEQDSKDSTYSLSST | |
| | LTLSKADYEKHKVYACEVTHQGLSSPVTKSFNRGEC | |
| 58 | PDTVTGRFTISRDNAKNTLYLEMSSLRSEDTAMYYCASRAYWG | 1963 |
| | QGTLVTVSAASTKGPSVFPLAPSSKSTSGGTAALGCLVKDYFPEP | Chimeric |
| | VTVSWNSGALTSGVHTFPAVLQSSGLYSLSSVVTVPSSSLGTQT | Heavy Chain |
| | YICNVNHKPSNTKVDKRVEPKSCDKTHTCPPCPAPELLGGPSVF | |
| | LFPPKPKDTLMISRTPEVTCVVVDVSHEDPEVKFNWYVDGVEV | |
| | HNAKTKPREEQYNSTYRVVSVLTVLHQDWLNGKEYKCKVSNK | |
| | ALPAPIEKTISKAKGQPREPQVYTLPPSREEMTKNQVSLTCLVKG | |
| | FYPSDIAVEWESNGQPENNYKTTPPVLDSDGSFFLYSKLTVDKS | |
| | RWQQGNVFSCSVMHEALHNHYTQKSLSLSPG | |
| 59 | DVLMTQTPLSLPVSLGDQASISCRSSQSIVHSNGNTYFEWYLQKP | 1963 |
| | GQSPKLLIYKVSNRFSGVPDRFSGSGSGTDFTLKISRVEAEDLGV | Chimeric |
| | YYCFQGSHVPYTFGGGTKLEIKRTVAAPSVFIFPPSDEQLKSGTA | Light Chain |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 60 | EVQLVESGGGLVKPGGSLRLSCAASGFTFSSYAMSWVRQAPGK | 1963-11 |
| | GLEWVSEISGGGSYTYYPDTVTGRFTISRDNAKNSLYLQMNSLR | Heavy Chain |
| | AEDTAVYYCASRAYWGQGTLVTVSSASTKGPSVFPLAPSSKSTS | |
| | GGTAALGCLVKDYFPEPVTVSWNSGALTSGVHTFPAVLQSSGL | |
| | YSLSSVVTVPSSSLGTQTYICNVNHKPSNTKVDKRVEPKSCDKT | |
| | HTCPPCPAPELLGGPSVFLFPPKPKDTLMISRTPEVTCVVVDVSH | |
| | EDPEVKFNWYVDGVEVHNAKTKPREEQYNSTYRVVSVLTVLH | |
| | QDWLNGKEYKCKVSNKALPAPIEKTISKAKGQPREPQVYTLPPS | |
| | REEMTKNQVSLTCLVKGFYPSDIAVEWESNGQPENNYKTTPPVL | |
| | DSDGSFFLYSKLTVDKSRWQQGNVFSCSVMHEALHNHYTQKSL | |
| | SLSPG | |
| 61 | EVQLVESGGGLVKPGGSLRLSCAASGFTFSSYAMSWVRQAPGK | 19G3-11 19 |
| | GLEWVSEISGGGSYTYYPDTVTGRFTISRDNAKNSLYLQMNSLR | |
| | AEDTAVYYCASRAYWGQGTLVTVSSASTKGPSVFPLAPSSKSTS | |
| | GGTAALGCLVKDYFPEPVTVSWNSGALTSGVHTFPAVLQSSGL | |
| | YSLSSVVTVPSSSLGTQTYICNVNHKPSNTKVDKRVEPKSCDKT | |
| | HTCPPCPAPELLGDDSVFLFPPKPKDTLMISRTPEVTCVVVDVSH | |
| | EDGEVKFNWYVDGVEVHNAKTKPREEQYNSTYRVVSVLTVLH | |
| | QDWLNGKEYKCKVSNKALPRPIEKTISKAKGQPREPQVYTLPPS | |
| | REEMTKNQVSLTCLVKGFYPSDIAVEWESNGQPENNYKTTPPVL | |
| | DSDGSFFLYSKLTVDKSRWQQGNVFSCSVMHEALHNHYTQKSL | |
| | SLSPG | |
| 62 | DIVMTQTPLSLPVTPGEPASISCRSSQSIVHSNGNTYFEWYLQKP | 1963-11 |
| | GQSPQLLIYKVSNRFSGVPDRFSGSGSGTDFTLKISRVEAEDVGV | Light Chain |
| | YYCFQGSHVPYTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 63 | EVQLVESGGGLVKPGGSLRLSCAASGFTFSSYAMSWVRQAPGK | 1963-22 |
| | GLEWVSEISGGGSYTYYPDTVTGRFTISRDNAKNSLYLQMNSLR | Heavy Chain |
| | AEDTAVYYCASRAYWGQGTLVTVSSASTKGPSVFPLAPSSKSTS | |
| | GGTAALGCLVKDYFPEPVTVSWNSGALTSGVHTFPAVLQSSGL | |
| | YSLSSVVTVPSSSLGTQTYICNVNHKPSNTKVDKRVEPKSCDKT | |
| | HTCPPCPAPELLGGPSVFLFPPKPKDTLMISRTPEVTCVVVDVSH | |
| | EDPEVKFNWYVDGVEVHNAKTKPREEQYNSTYRVVSVLTVLH | |
| | QDWLNGKEYKCKVSNKALPAPIEKTISKAKGQPREPQVYTLPPS | |
| | REEMTKNQVSLTCLVKGFYPSDIAVEWESNGQPENNYKTTPPVL | |
| | DSDGSFFLYSKLTVDKSRWQQGNVFSCSVMHEALHNHYTQKSL | |
| | SLSPG | |
| 64 | DIVMTQTPLSLPVTPGEPASISCRSSQSIVHSQGNTYFEWYLQKP | 1963-22 |
| | GQSPQLLIYKVSNRFSGVPDRFSGSGSGTDFTLKISRVEAEDVGV | Light Chain |
| | YYCFQGSHVPYTFGGGTKVEIKRTVAAPSVFIFPPSDEQLKSGTA | |
| | SVVCLLNNFYPREAKVQWKVDNALQSGNSQESVTEQDSKDSTY | |
| | SLSSTLTLSKADYEKHKVYACEVTHQGLSSPVTKSFNRGEC | |
| 65 | ASTKGPSVFPLAPSSKSTSGGTAALGCLVKDYFPEPVTVSWNSG | Human |
| | ALTSGVHTFPAVLQSSGLYSLSSVVTVPSSSLGTQTYICNVNHKP | Constant |
| | SNTKVDKRVEPKSCDKTHTCPPCPAPELLGGPSVFLFPPKPKDTL | Domain |
| | MISRTPEVTCVVVDVSHEDPEVKFNWYVDGVEVHNAKTKPREE | IgGif |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| 66 | ASTKGPSVFPLAPSSKSTSGGTAALGCLVKDYFPEPVTVSWNSG | SE Constant |
| | ALTSGVHTFPAVLQSSGLYSLSSVVTVPSSSLGTQTYICNVNHKP | Domain |
| | SNTKVDKRVEPKSCDKTHTCPPCPAPELLGGPSVFLFPPKPKDTL | |
| | MISRTPEVTCVVVDVEHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| 67 | ASTKGPSVFPLAPSSKSTSGGTAALGCLVKDYFPEPVTVSWNSG | SELF |
| | ALTSGVHTFPAVLQSSGLYSLSSVVTVPSSSLGTQTYICNVNHKP | Constant |
| | SNTKVDKRVEPKSCDKTHTCPPCPAPELLGGPSVFLFPPKPKDTL | Domain |
| | MISRTPEVTCVVVDVEHEDPEVKFNWYVDGVEVHNAKTKPREE | |
| | QYNSTYRVVSVLTVLHQDWLNGKEYKCKVSNKAFPAPIEKTISK | |
| | AKGQPREPQVYTLPPSREEMTKNQVSLTCLVKGFYPSDIAVEWE | |
| | SNGQPENNYKTTPPVLDSDGSFFLYSKLTVDKSRWQQGNVFSCS | |
| | VMHEALHNHYTQKSLSLSPG | |
| 68 | ASTKGPSVFPLAPSSKSTSGGTAALGCLVKDYFPEPVTVSWNSG | P238D |
| | ALTSGVHTFPAVLQSSGLYSLSSVVTVPSSSLGTQTYICNVNHKP | Constant |
| | SNTKVDKRVEPKSCDKTHTCPPCPAPELLGGDSVFLFPPKPKDTL | Domain |
| | MISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| 69 | ASTKGPSVFPLAPSSKSTSGGTAALGCLVKDYFPEPVTVSWNSG | V4 Constant |
| | ALTSGVHTFPAVLQSSGLYSLSSVVTVPSSSLGTQTYICNVNHKP | Domain |
| | SNTKVDKRVEPKSCDKTHTCPPCPAPELLGGDSVFLFPPKPKDTL | |
| | MISRTPEVTCVVVDVSHEDGEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| 70 | ASTKGPSVFPLAPSSKSTSGGTAALGCLVKDYFPEPVTVSWNSG | V4 D270E |
| | ALTSGVHTFPAVLQSSGLYSLSSVVTVPSSSLGTQTYICNVNHKP | Constant |
| | SNTKVDKRVEPKSCDKTHTCPPCPAPELLGGDSVFLFPPKPKDTL | Domain |
| | MISRTPEVTCVVVDVSHEEGEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| 71 | ASTKGPSVFPLAPSSKSTSGGTAALGCLVKDYFPEPVTVSWNSG | V7 Constant |
| | ALTSGVHTFPAVLQSSGLYSLSSVVTVPSSSLGTQTYICNVNHKP | Domain |
| | SNTKVDKRVEPKSCDKTHTCPPCPAPDLLGGDSVFLFPPKPKDT | |
| | LMISRTPEVTCVVVDVSHEDGEVKFNWYVDGVEVHNAKTKPRE | |
| | EQYNSTYRVVSVLTVLHQDWLNGKEYKCKVSNKALPRPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| 72 | ASTKGPSVFPLAPSSKSTSGGTAALGCLVKDYFPEPVTVSWNSG | V8 Constant |
| | ALTSGVHTFPAVLQSSGLYSLSSVVTVPSSSLGTQTYICNVNHKP | Domain |
| | SNTKVDKRVEPKSCDKTHTCPPCPAPELLGDDSVFLFPPKPKDTL | |
| | MISRTPEVTCVVVDVSDEDGEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| 73 | ASTKGPSVFPLAPSSKSTSGGTAALGCLVKDYFPEPVTVSWNSG | V9 Constant |
| | ALTSGVHTFPAVLQSSGLYSLSSVVTVPSSSLGTQTYICNVNHKP | Domain |
| | SNTKVDKRVEPKSCDKTHTCPPCPAPELLGDDSVFLFPPKPKDTL | |
| | MISRTPEVTCVVVDVSHEDGEVKFNWYVDGVEVHNAKTKPREE | |
| | QYNSTYRVVSVLTVLHQDWLNGKEYKCKVSNKALPRPIEKTISK | |
| | AKGQPREPQVYTLPPSREEMTKNQVSLTCLVKGFYPSDIAVEWE | |
| | SNGQPENNYKTTPPVLDSDGSFFLYSKLTVDKSRWQQGNVFSCS | |
| | VMHEALHNHYTQKSLSLSPG | |
| 74 | ASTKGPSVFPLAPSSKSTSGGTAALGCLVKDYFPEPVTVSWNSG | V9 D270E |
| | ALTSGVHTFPAVLQSSGLYSLSSVVTVPSSSLGTQTYICNVNHKP | Constant |
| | SNTKVDKRVEPKSCDKTHTCPPCPAPELLGDDSVFLFPPKPKDTL | Domain |
| | MISRTPEVTCVVVDVSHEEGEVKFNWYVDGVEVHNAKTKPREE | |
| | QYNSTYRVVSVLTVLHQDWLNGKEYKCKVSNKALPRPIEKTISK | |
| | AKGQPREPQVYTLPPSREEMTKNQVSLTCLVKGFYPSDIAVEWE | |
| | SNGQPENNYKTTPPVLDSDGSFFLYSKLTVDKSRWQQGNVFSCS | |
| | VMHEALHNHYTQKSLSLSPG | |
| 75 | ASTKGPSVFPLAPSSKSTSGGTAALGCLVKDYFPEPVTVSWNSG | V11 |
| | ALTSGVHTFPAVLQSSGLYSLSSVVTVPSSSLGTQTYICNVNHKP | Constant |
| | SNTKVDKRVEPKSCDKTHTCPPCPAPELLGDDSVFLFPPKPKDTL | Domain |
| | MISRTPEVTCVVVDVSDEDGEVKFNWYVDGVEVHNAKTKPREE | |
| | QYNSTYRVVSVLTVLHQDWLNGKEYKCKVSNKALPRPIEKTISK | |
| | AKGQPREPQVYTLPPSREEMTKNQVSLTCLVKGFYPSDIAVEWE | |
| | SNGQPENNYKTTPPVLDSDGSFFLYSKLTVDKSRWQQGNVFSCS | |
| | VMHEALHNHYTQKSLSLSPG | |
| 76 | ASTKGPSVFPLAPSSKSTSGGTAALGCLVKDYFPEPVTVSWNSG | V12 |
| | ALTSGVHTFPAVLQSSGLYSLSSVVTVPSSSLGTQTYICNVNHKP | Constant |
| | SNTKVDKRVEPKSCDKTHTCPPCPAPDLLGDDSVFLFPPKPKDT | Domain |
| | LMISRTPEVTCVVVDVSDEDGEVKFNWYVDGVEVHNAKTKPRE | |
| | EQYNSTYRVVSVLTVLHQDWLNGKEYKCKVSNKALPRPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| 77 | RTVAAPSVFIFPPSDEQLKSGTASVVCLLNNFYPREAKVQWKVD | Light Chain |
| | NALQSGNSQESVTEQDSKDSTYSLSSTLTLSKADYEKHKVYACE | Kappa |
| | VTHQGLSSPVTKSFNRGEC | Constant |
| | | Domain |
| 78 | MRAWIFFLLCLAGRALA | Signal |
| | | Sequence |
| 79 | QVQLVQSGAEVKKPGASVKVSCKASGYTFTGYYMHWVRQAPG | 2141-IgG1 |
| | QGLEWMGWINPDSGGTNYAQKFQGRVTMTRDTSISTAYMELN | Heavy chain |
| | RLRSDDTAVYYCARDQPLGYCTNGVCSYFDYWGQGTLVTVSS | |
| | ATTKGPSVFPLAPSSKSTSGGTAALGCLVKDYFPEPVTVSWNSG | |
| | ALTSGVHTFPAVLQSSGLYSLSSVVTVPSSSLGTQTYICNVNHKP | |
| | SNTKVDKRVEPKSCDKTHTCPPCPAPELLGGPSVFLFPPKPKDTL | |
| | MISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSRDELTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPGK | |
| 80 | DIQMTQSPSSVSASVGDRVTITCRASQGIYSWLAWYQQKPGKAP | 2141-IgG1 |
| | NLLIYTASTLQSGVPSRFSGSGSGTDFTLTISSLQPEDFATYYCQQ | Light chain |
| | ANIFPLTFGGGTKVEIKRTVAAPSVFIFPPSDEQLKSGTASVVCLL | |
| | NNFYPREAKVQWKVDNALQSGNSQESVTEQDSKDSTYSLSSTL | |
| | TLSKADYEKHKVYACEVTHQGLSSPVTKSFNRGEC | |
| 81 | QVQLVQSGAEVKKPGASVKVSCKASGYTFTGYYMHWVRQAPG | 2141-IgG1 |
| | QGLEWMGWINPDSGGTNYAQKFQGRVTMTRDTSISTAYMELN | N297A |
| | RLRSDDTAVYYCARDQPLGYCTNGVCSYFDYWGQGTLVTVSS | Heavy chain |
| | ATTKGPSVFPLAPSSKSTSGGTAALGCLVKDYFPEPVTVSWNSG | |
| | ALTSGVHTFPAVLQSSGLYSLSSVVTVPSSSLGTQTYICNVNHKP | |
| | SNTKVDKRVEPKSCDKTHTCPPCPAPELLGGPSVFLFPPKPKDTL | |
| | MISRTPEVTCVVVDVSHEDPEVKFNWYVDGVEVHNAKTKPREE | |
| | QYASTYRVVSVLTVLHQDWLNGKEYKCKVSNKALPAPIEKTIS | |
| | KAKGQPREPQVYTLPPSRDELTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPGK | |
| 82 | QVQLVQSGAEVKKPGASVKVSCKASGYTFTGYYMHWVRQAPG | 2141-IgG1 |
| | QGLEWMGWINPDSGGTNYAQKFQGRVTMTRDTSISTAYMELN | S267E (SE) |
| | RLRSDDTAVYYCARDQPLGYCTNGVCSYFDYWGQGTLVTVSS | Heavy chain |
| | ATTKGPSVFPLAPSSKSTSGGTAALGCLVKDYFPEPVTVSWNSG | |
| | ALTSGVHTFPAVLQSSGLYSLSSVVTVPSSSLGTQTYICNVNHKP | |
| | SNTKVDKRVEPKSCDKTHTCPPCPAPELLGGPSVFLFPPKPKDTL | |
| | MISRTPEVTCVVVDVEHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSRDELTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPGK | |
| 83 | QVQLVQSGAEVKKPGASVKVSCKASGYTFTGYYMHWVRQAPG | 2141-IgG1 |
| | QGLEWMGWINPDSGGTNYAQKFQGRVTMTRDTSISTAYMELN | S267E/L328F |
| | RLRSDDTAVYYCARDQPLGYCTNGVCSYFDYWGQGTLVTVSS | (SELF) |
| | ATTKGPSVFPLAPSSKSTSGGTAALGCLVKDYFPEPVTVSWNSG | Heavy chain |
| | ALTSGVHTFPAVLQSSGLYSLSSVVTVPSSSLGTQTYICNVNHKP | |
| | SNTKVDKRVEPKSCDKTHTCPPCPAPELLGGPSVFLFPPKPKDTL | |
| | MISRTPEVTCVVVDVEHEDPEVKFNWYVDGVEVHNAKTKPREE | |
| | QYNSTYRVVSVLTVLHQDWLNGKEYKCKVSNKAFPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSCS | |
| | VMHEALHNHYTQKSLSLSPGK | |
| 84 | QVQLVQSGAEVKKPGASVKVSCKASGYTFTGYYMHWVRQAPG | 2141-IgG1 |
| | QGLEWMGWINPDSGGTNYAQKFQGRVTMTRDTSISTAYMELN | G237D/P238D/ |
| | RLRSDDTAVYYCARDQPLGYCTNGVCSYFDYWGQGTLVTVSS | P271G/ |
| | ATTKGPSVFPLAPSSKSTSGGTAALGCLVKDYFPEPVTVSWNSG | A330R (V9) |
| | ALTSGVHTFPAVLQSSGLYSLSSVVTVPSSSLGTQTYICNVNHKP | Heavy chain |
| | SNTKVDKRVEPKSCDKTHTCPPCPAPELLGDDSVFLFPPKPKDTL | |
| | MISRTPEVTCVVVDVSHEDGEVKFNWYVDGVEVHNAKTKPREE | |
| | QYNSTYRVVSVLTVLHQDWLNGKEYKCKVSNKALPRPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSCS | |
| | VMHEALHNHYTQKSLSLSPGK | |
| 85 | QVQLVQSGAEVKKPGASVKVSCKASGYTFTGYYMHWVRQAPG | 2141-IgG1 |
| | QGLEWMGWINPDSGGTNYAQKFQGRVTMTRDTSISTAYMELN | G237D/P238D/ |
| | RLRSDDTAVYYCARDQPLGYCTNGVCSYFDYWGQGTLVTVSS | H268D/P271G/ |
| | ATTKGPSVFPLAPSSKSTSGGTAALGCLVKDYFPEPVTVSWNSG | A330R |
| | ALTSGVHTFPAVLQSSGLYSLSSVVTVPSSSLGTQTYICNVNHKP | (V11) |
| | SNTKVDKRVEPKSCDKTHTCPPCPAPELLGDDSVFLFPPKPKDTL | Heavy chain |
| | MISRTPEVTCVVVDVSDEDGEVKFNWYVDGVEVHNAKTKPREE | |
| | QYNSTYRVVSVLTVLHQDWLNGKEYKCKVSNKALPRPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSCS | |
| | VMHEALHNHYTQKSLSLSPGK | |
| 86 | QVQLVQSGAEVKKPGASVKVSCKASGYTFTGYYMHWVRQAPG | 2141-IgG2- |
| | QGLEWMGWINPDSGGTNYAQKFQGRVTMTRDTSISTAYMELN | Heavy chain |
| | RLRSDDTAVYYCARDQPLGYCTNGVCSYFDYWGQGTLVTVSS | |
| | ASTKGPSVFPLAPCSRSTSESTAALGCLVKDYFPEPVTVSWNSGA | |
| | LTSGVHTFPAVLQSSGLYSLSSVVTVPSSNFGTQTYTCNVDHKPS | |
| | NTKVDKTVERKCCVECPPCPAPPVAGPSVFLFPPKPKDTLMISRT | |
| | PEVTCVVVDVSHEDPEVQFNWYVDGVEVHNAKTKPREEQFNST | |
| | FRVVSVLTVVHQDWLNGKEYKCKVSNKGLPAPIEKTISKTKGQP | |
| | REPQVYTLPPSREEMTKNQVSLTCLVKGFYPSDIAVEWESNGQP | |
| | ENNYKTTPPMLDSDGSFFLYSKLTVDKSRWQQGNVFSCSVMHE | |
| | ALHNHYTQKSLSLSPGK | |
| 87 | QVQLVQSGAEVKKPGASVKVSCKASGYTFTGYYMHWVRQAPG | 2141-IgG2 |
| | QGLEWMGWINPDSGGTNYAQKFQGRVTMTRDTSISTAYMELN | C127S- |
| | RLRSDDTAVYYCARDQPLGYCTNGVCSYFDYWGQGTLVTVSS | Heavy chain |
| | ASTKGPSVFPLAPSSRSTSESTAALGCLVKDYFPEPVTVSWNSGA | |
| | LTSGVHTFPAVLQSSGLYSLSSVVTVPSSNFGTQTYTCNVDHKPS | |
| | NTKVDKTVERKCCVECPPCPAPPVAGPSVFLFPPKPKDTLMISRT | |
| | PEVTCVVVDVSHEDPEVQFNWYVDGVEVHNAKTKPREEQFNST | |
| | FRVVSVLTVVHQDWLNGKEYKCKVSNKGLPAPIEKTISKTKGQP | |
| | REPQVYTLPPSREEMTKNQVSLTCLVKGFYPSDIAVEWESNGQP | |
| | ENNYKTTPPMLDSDGSFFLYSKLTVDKSRWQQGNVFSCSVMHE | |
| | ALHNHYTQKSLSLSPGK | |
| 88 | QVQLVQSGAEVKKPGASVKVSCKASGYTFTGYYMHWVRQAPG | 2141-IgG2 |
| | QGLEWMGWINPDSGGTNYAQKFQGRVTMTRDTSISTAYMELN | C232S- |
| | RLRSDDTAVYYCARDQPLGYCTNGVCSYFDYWGQGTLVTVSS | Heavy chain |
| | ASTKGPSVFPLAPCSRSTSESTAALGCLVKDYFPEPVTVSWNSGA | |
| | LTSGVHTFPAVLQSSGLYSLSSVVTVPSSNFGTQTYTCNVDHKPS | |
| | NTKVDKTVERKSCVECPPCPAPPVAGPSVFLFPPKPKDTLMISRT | |
| | PEVTCVVVDVSHEDPEVQFNWYVDGVEVHNAKTKPREEQFNST | |
| | FRVVSVLTVVHQDWLNGKEYKCKVSNKGLPAPIEKTISKTKGQP | |
| | REPQVYTLPPSREEMTKNQVSLTCLVKGFYPSDIAVEWESNGQP | |
| | ENNYKTTPPMLDSDGSFFLYSKLTVDKSRWQQGNVFSCSVMHE | |
| | ALHNHYTQKSLSLSPGK | |

In some embodiments, the anti-CD40 antibody or antigen binding fragment thereof comprises three heavy chain complementarity determining regions (HCDR1, HCDR2, and HCDR3) of a heavy chain having an amino acid sequence selected from the group consisting of SEQ ID NOs:79, 81-88 and three light chain complementarity determining regions (LCDR1, LCDR2, and LCDR3) of a light chain having the amino acid sequence of SEQ ID NO: 80.

In some embodiments, the anti-CD40 antibody or antigen binding fragment thereof comprises a heavy chain variable region (HCVR) of a heavy chain having an amino acid sequence selected from the group consisting of SEQ ID NOs:79, 81-88 and a light chain variable region (LCVR) of a light chain having the amino acid sequence of SEQ ID NO: 80.

In some embodiments, the anti-CD40 antibody or antigen binding fragment thereof comprises an Fc region modified to enhance specificity of binding to FcγRIIb, wherein the Fc region comprises a sequence selected from the group consisting of SE (SEQ ID NO: 66), SELF (SEQ ID NO: 67), P238D (SEQ ID NO: 68), V4 (SEQ ID NO: 69), V4 D270E (SEQ ID NO: 70), V7 (SEQ ID NO: 71), V8 (SEQ ID NO: 72), V9 (SEQ ID NO: 73), V9 D270E (SEQ ID NO: 74), V11 (SEQ ID NO: 75), and V12 (SEQ ID NO: 76).

In some embodiments, the anti-CD40 antibody or antigen binding fragment thereof comprises a heavy chain having an amino acid sequence selected from the group consisting of SEQ ID NO: 79, 81-88 and a light chain having the amino acid sequence of SEQ ID NO: 80.

In some embodiments, the treatment produces a therapeutic effect selected from one or more of: delay in tumor growth, reduction in tumor cell number, tumor regression, prevention or delay of tumor recurrence, increase in survival, partial response, and complete response.

In some embodiments, the tumor growth is inhibited by at least 50% as compared to an untreated patient. In some embodiments, the tumor growth is inhibited by at least 50% as compared to a patient administered the anti-CD40 antibody or antigen binding fragment thereof or the IL-15 polypeptide as a monotherapy.

In another aspect, this disclosure provides a combination of an anti-CD40 antibody or antigen binding fragment thereof or an IL-15 polypeptide for use in a method of treating a cancer. The method comprises: selecting a patient with a cancer; and administering to the patient in need thereof a therapeutically effective amount of anti-CD40 antibody or antigen binding fragment thereof in combination with a therapeutically effective amount of the IL-15 polypeptide, wherein the anti-CD40 antibody is selected from 2141-V11, CP-870,893-V11, ChiLob7/6-V11, 12D6-V11, 5F11-V11, 8E8-V11, 5G7-V11, 19G3-V11, APX005M-V11, ADC-1013-V11, CDX-1140-V11, SEA-CD40-V11, SGN-CD40-V11, and ABBV-927-V11.

In some embodiments, the 2141-V11 comprises a heavy chain having an amino acid sequence selected from the group consisting of SEQ ID NOs:79, 81-88 and a light chain having the amino acid sequence of SEQ ID NO: 80.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the VL, VH, CL, and CHI domains; (ii) a F(ab′)2 fragment, a bivalent fragment comprising two Fab fragments linked by a disulfide bridge at the hinge region; (iii) a Fab′ fragment, which is essentially a Fab with part of the hinge region (see, FUNDAMENTAL IMMUNOLOGY (Paul ed., 3rd ed. 1993)); (iv) a Fd fragment consisting of the VH and CHI domains; (v) a Fv fragment consisting of the VL and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities. An isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of this disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created, or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species, and the constant region sequences are derived from another species, such as an antibody in which the variable region sequences are derived from a mouse antibody, and the constant region sequences are derived from a human antibody. The term can also refer to an antibody in which its variable region sequence or CDR(s) is derived from one source (e.g., an IgA1 antibody) and the constant region sequence or Fc is derived from a different source (e.g., a different antibody, such as an IgG, IgA2, IgD, IgE or IgM antibody).

### Fragment

In some embodiments, an antibody provided herein is an antibody fragment. Antibody fragments include, but are not limited to, Fab, Fab′, Fab′-SH, F(ab′)2, Fv, and single-chain Fv (scFv) fragments, and other fragments described below, e.g., diabodies, triabodies tetrabodies, and single-domain antibodies. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (DOMANTIS, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### Chimeric and Humanized Antibodies

In some embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In some embodiments, an antibody provided herein is a human antibody. Human antibodies can be produced using various techniques known in the art or using techniques described herein. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example, U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

### Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

### Substitution, Insertion, and Deletion Variants

In some embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved antibody-dependent cell-mediated cytotoxicity (ADCC) and complement-dependent cytotoxicity (CDC).

Accordingly, an antibody of the disclosure can comprise one or more conservative modifications of the CDRs, heavy chain variable region, or light variable regions described herein. A conservative modification or functional equivalent of a peptide, polypeptide, or protein disclosed in this disclosure refers to a polypeptide derivative of the peptide, polypeptide, or protein, e.g., a protein having one or more point mutations, insertions, deletions, truncations, a fusion protein, or a combination thereof. It substantially retains the activity of the parent peptide, polypeptide, or protein (such as those disclosed in this disclosure). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent. Accordingly, within the scope of this disclosure are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

As used herein, the term “conservative modifications” refers to amino acid modifications that do not significantly affect or alter the binding characteristics of the antibody containing the amino acid sequence. Such conservative modifications include amino acid substitutions, additions and deletions. Modifications can be introduced into an antibody of this disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: (i) amino acids with basic side chains (e.g., lysine, arginine, histidine), (ii) acidic side chains (e.g., aspartic acid, glutamic acid), (iii) uncharged polar side chains (e.g., glycine, asparagine, glutamine, serine, threonine, tyrosine, cysteine, tryptophan), (iv) nonpolar side chains (e.g., alanine, valine, leucine, isoleucine, proline, phenylalanine, methionine), (v) beta-branched side chains (e.g., threonine, valine, isoleucine), and (vi) aromatic side chains (e.g., tyrosine, phenylalanine, tryptophan, histidine).

Non-conservative substitutions will entail exchanging a member of one of these classes for another class.

An exemplary substitutional variant is an affinity matured antibody, which may be conveniently generated, e.g., using phage display-based affinity maturation techniques such as those described in, e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites are created or removed.

For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyltransferase, such that antibodies expressed in such a cell line exhibit hypofucosylation. PCT Publication WO 03/035835 by Presta describes a variant Chinese Hamster Ovary cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyltransferases (e.g., beta(1,4)-N-acetylglucosaminyltransferase III (GnTIII)) such that antibodies expressed in the engineered cell lines exhibit increased bisecting GlcNac structures which result in increased ADCC activity of the antibodies (see also Umana et al. (1999) Nat. Biotech. 17: 176-180).

### Fc Region Variants

The variable regions of the antibody described herein can be linked (e.g., covalently linked or fused) to an Fc, e.g., an IgG1, IgG2, IgG3 or IgG4 Fc, which may be of any allotype or isoallotype, e.g., for IgG1: Glm, Glm1(a), Glm2(x), Glm3(f), Glm17(z); for IgG2: G2m, G2m23(n); for IgG3: G3m, G3m21(g1), G3m28(g5), G3 ml 1(b0), G3m5(b1), G3m13(b3), G3m14(b4), G3m10(b5), G3m15(s), G3m16(t), G3m6(c3), G3m24(c5), G3m26(u), G3m27(v); and for K: Km, Km1, Km2, Km3 (see, e.g., Jefferies et al. (2009) mAbs 1:1). In some embodiments, the antibodies variable regions described herein are linked to an Fe that binds to one or more activating Fc receptors (FcγI, FcγIIa or FcγIIIa), and thereby stimulate ADCC and may cause T cell depletion. In some embodiments, the antibody variable regions described herein are linked to an Fc that causes depletion.

In some embodiments, the antibody variable regions described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE and IgM. The constant region of an immunoglobulin is defined as a naturally-occurring or synthetically-produced polypeptide homologous to the immunoglobulin C-terminal region and can include a CH1 domain, a hinge, a CH2 domain, a CH3 domain, or a CH4 domain, separately or in combination. In some embodiments, an antibody of this disclosure has an Fc region other than that of a wild type IgA1. The antibody can have an Fc region from that of IgG (e.g., IgG1, IgG2, IgG3, and IgG4) or other classes such as IgA2, IgD, IgE, and IgM. The Fc can be a mutant form of IgA1.

The constant region of an immunoglobulin is responsible for many important antibody functions, including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgG1, IgG2, IgG3, and IgG4.

Ig molecules interact with multiple classes of cellular receptors. For example, IgG molecules interact with three classes of Fcγ receptors (FcγR) specific for the IgG class of antibody, namely FcγRI, FcγRII, and FcγRIIL. The important sequences for the binding of IgG to the FcγR receptors have been reported to be located in the CH2 and CH3 domains. The serum half-life of an antibody is influenced by the ability of that antibody to bind to an FcR.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased CDC, (c) has increased or decreased affinity for Clq and/or (d) has increased or decreased affinity for an Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc., substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli, such as proline iminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the Clq binding site, may be removed from the Fc region. For example, one may delete or substitute the EKK sequence of human IgG1. In some embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fc domains are disclosed, for example, in WO 97/34631 and WO 96/32478.

In one embodiment, the hinge region of Fe is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260, both by Winter et al.

In another example, one or more amino acids selected from amino acid residues 329, 331, and 322 can be replaced with a different amino acid residue such that the antibody has altered Clq binding and/or reduced or abolished CDC. This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase ADCC and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in abat (WO00/42072).

Other Fc modifications that can be made to Fcs are those for reducing or ablating binding to FcγR and/or complement proteins, thereby reducing or ablating Fc-mediated effector functions such as ADCC, antibody-dependent cellular phagocytosis (ADCP), and CDC. Exemplary modifications include but are not limited to substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; WO00/42072; WO01/58957; WO02/06919; WO04/016750; WO04/029207; WO04/035752; WO04/074455; WO04/099249; WO04/063351; WO05/070963; WO05/040217, WO05/092925, and WO06/020114).

Fc variants that enhance affinity for an inhibitory receptor FcγRIIb may also be used. Such variants may provide an Fc fusion protein with immune-modulatory activities related to FcγRIIb cells, including, for example, B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art, including but not limited to, equilibrium methods (e.g., ELISA, or radioimmunoassay), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods, including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In some embodiments, the antibody is modified to increase its biological half-life. Various approaches are possible. For example, this may be done by increasing the binding affinity of the Fc region for FcRn. For example, one or more of the following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In some embodiments, hybrid IgG isotypes with particular biological characteristics may be used. For example, an IgG1/IgG3 hybrid variant may be constructed by substituting IgG 1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 422I, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed chat comprises one or more substitutions, e.g., one or more of the following amino acid substitutions: 233E, 234L, 235L, 236G (referring to an insertion of a glycine at position 236), and 321 h.

Moreover, the binding sites on human IgG1 for FcγRI, FcγRII, FcγRIII, and FcRn have been mapped and variants with improved binding have been described (see Shields, R. L. et al. (2001) J. Biol. Chem. 276:6591-6604). Specific mutations at positions 256, 290, 298, 333, 334, and 339 were shown to improve binding to FcγRIII. Additionally, the following combination mutants were shown to improve FcγRIII binding: T256A/S298A, S298A/E333A, S298A/K224A, and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52−specific), trastuzumab (HER2/neu− specific), rituximab (CD20−specific), and cetuximab (EGFR− specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/I332E, S239D/I332E/A330L, L235V/F243L/R292P/Y300L/P396L, and M428L/N434S.

In some embodiments, an Fc is chosen that has reduced binding to FcγRs. An exemplary Fc, e.g., IgG1 Fc, with reduced FcγR binding, comprises the following three amino acid substitutions: L234A, L235E, and G237A. In some embodiments, an Fc is chosen that has reduced complement fixation. An exemplary Fc, e.g., IgG1 Fc, with reduced complement fixation, has the following two amino acid substitutions: A330S and P331S. In some embodiments, an Fc is chosen that has essentially no effector function, i.e., it has reduced binding to FcγRs and reduced complement fixation. An exemplary Fc, e.g., IgG1 Fc, that is effectorless, comprises the following five mutations: L234A, L235E, G237A, A330S, and P331S. When using an IgG4 constant domain, it is usually preferable to include the substitution S228P, which mimics the hinge sequence in IgG1 and thereby stabilizes IgG4 molecules.

### Multivalent Antibodies

In one embodiment, the antibodies of this disclosure may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0129125. In each case, at least one of the binding sites will comprise an epitope, motif or domain associated with a DLL3 isoform.

In one embodiment, the antibodies are bispecific antibodies in which the two chains have different specificities, as described in Millstein et al., 1983, Nature, 305:537-539. Other embodiments include antibodies with additional specificities, such as tri-specific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As stated above, multivalent antibodies may immunospecifically bind to different epitopes of the desired target molecule or may immunospecifically bind to both the target molecule as well as a heterologous epitope, such as a heterologous polypeptide or solid support material. In some embodiments, the multivalent antibodies may include bispecific antibodies or trispecific antibodies. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art and are disclosed in U.S. Pat. No. 4,676,980, along with a number of cross-linking techniques.

In some embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

### Antibody Derivatives

An antibody provided herein may be further modified to contain additional nonproteinaceous moieties that are known in the art and readily available. The moieties suitable for derivatization of the antibody include but are not limited to water-soluble polymers.

Non-limiting examples of water-soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies described herein. See, for example, EP 0 154 316 by Nishimura et al. and EP0401384 by Ishikawa et al.

The present disclosure also encompasses a human monoclonal antibody described herein conjugated to a therapeutic agent, a polymer, a detectable label or enzyme. In one embodiment, the therapeutic agent is a cytotoxic agent. In one embodiment, the polymer is PEG.

### Nucleic Acids, Expression Cassettes, and Vectors

The present disclosure provides isolated nucleic acid segments that encode the polypeptides, peptide fragments, and coupled proteins of this disclosure. The nucleic acid segments of this disclosure also include segments that encode for the same amino acids due to the degeneracy of the genetic code. For example, the amino acid threonine is encoded by ACU, ACC, ACA, and ACG and is therefore degenerate. It is intended that the disclosure includes all variations of the polynucleotide segments that encode for the same amino acids. Such mutations are known in the art (Watson et al., Molecular Biology of the Gene, Benjamin Cummings 1987). Mutations also include alteration of a nucleic acid segment to encode for conservative amino acid changes, for example, the substitution of leucine for isoleucine and so forth. Such mutations are also known in the art. Thus, the genes and nucleotide sequences of this disclosure include both the naturally occurring sequences as well as mutant forms.

The nucleic acid segments of this disclosure may be contained within a vector. A vector may include, but is not limited to, any plasmid, phagemid, F-factor, virus, cosmid, or phage in a double- or single-stranded linear or circular form which may or may not be self transmissible or mobilizable. The vector can also transform a prokaryotic or eukaryotic host either by integration into the cellular genome or exist extra-chromosomally (e.g., autonomous replicating plasmid with an origin of replication).

Preferably the nucleic acid segment in the vector is under the control of, and operably linked to, an appropriate promoter or other regulatory elements for transcription in vitro or in a host cell, such as a eukaryotic cell, or a microbe, e.g., bacteria. The vector may be a shuttle vector that functions in multiple hosts. The vector may also be a cloning vector that typically contains one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech).

The nucleic acid segments of this disclosure may also be inserted into an expression vector. Typically an expression vector contains prokaryotic DNA elements coding for a bacterial replication origin and an antibiotic resistance gene to provide for the amplification and selection of the expression vector in a bacterial host; regulatory elements that control initiation of transcription such as a promoter; and DNA elements that control the processing of transcripts such as introns, or a transcription termination/polyadenylation sequence.

Methods to introduce nucleic acid segment into a vector are available in the art (Sambrook et al., Molecular Cloning: A Laboratory Manual, 3rd edition, Cold Spring Harbor Press, Cold Spring Harbor, N.Y. (2001)). Briefly, a vector into which a nucleic acid segment is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a nucleic acid segment into the vector. The nucleic acid segment that is to be inserted into the vector is treated with one or more restriction enzymes to create a linearized segment having a blunt end, a “sticky” end with a 5′ or a 3′ overhang, or any combination of the above. The nucleic acid segment may also be treated with a restriction enzyme and subsequently treated with another DNA modifying enzyme. Such DNA modifying enzymes include, but are not limited to, polymerase, exonuclease, phosphatase or a kinase, to create a nucleic acid segment that has characteristics useful for ligation of a nucleic acid segment into the vector.

The treated vector and nucleic acid segment are then ligated together to form a construct containing a nucleic acid segment according to methods available in the art (Sambrook et al., Molecular Cloning: A Laboratory Manual, 3rd edition, Cold Spring Harbor Press, Cold Spring Harbor, N.Y. (2001)). Briefly, the treated nucleic acid fragment, and the treated vector are combined in the presence of a suitable buffer and ligase. The mixture is then incubated under appropriate conditions to allow the ligase to ligate the nucleic acid fragment into the vector.

The disclosure also provides an expression cassette which contains a nucleic acid sequence capable of directing expression of a particular nucleic acid segment of this disclosure, either in vitro or in a host cell. Also, a nucleic acid segment of this disclosure may be inserted into the expression cassette such that an anti-sense message is produced. The expression cassette is an isolatable unit such that the expression cassette may be in linear form and functional for in vitro transcription and translation assays. The materials and procedures to conduct these assays are commercially available from Promega Corp. (Madison, Wis.). For example, an in vitro transcript may be produced by placing a nucleic acid sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a nucleic acid segment.

Such an expression cassette may contain one or a plurality of restriction sites allowing for placement of the nucleic acid segment under the regulation of a regulatory sequence. The expression cassette can also contain a termination signal operably linked to the nucleic acid segment as well as regulatory sequences required for proper translation of the nucleic acid segment. The expression cassette containing the nucleic acid segment may be chimeric, meaning that at least one of its components is heterologous with respect to at least one of its other components. The expression cassette may also be one that is naturally occurring but has been obtained in a recombinant form useful for heterologous expression. Expression of the nucleic acid segment in the expression cassette may be under the control of a constitutive promoter or an inducible promoter, which initiates transcription only when the host cell is exposed to some particular external stimulus.

The expression cassette may include in the 5′-3′ direction of transcription, a transcriptional and translational initiation region, a nucleic acid segment and a transcriptional and translational termination region functional in vivo and/or in vitro. The termination region may be native with the transcriptional initiation region, may be native with the nucleic acid segment, or may be derived from another source.

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences, which may be a combination of synthetic and natural sequences. While regulatory sequences are not limited to promoters, some useful regulatory sequences include constitutive promoters, inducible promoters, regulated promoters, tissue-specific promoters, viral promoters, and synthetic promoters.

A promoter is a nucleotide sequence that controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors that control the effectiveness of transcription initiation in response to physiological or developmental conditions.

The disclosure also provides a construct containing a vector and an expression cassette. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a nucleic acid segment of this disclosure into a vector that itself contains regulatory sequences. Thus, an expression cassette is formed upon insertion of the nucleic acid segment into the vector. Vectors containing regulatory sequences are available commercially, and methods for their use are known in the art (Clonetech, Promega, Stratagene).

In another aspect, this disclosure also provides (i) a nucleic acid molecule encoding a polypeptide chain of the antibody or antigen-binding fragment thereof described herein; (ii) a vector comprising the nucleic acid molecule as described; and (iii) a cultured host cell comprising the vector as described. Also provided is a method for producing a polypeptide, comprising: (a) obtaining the cultured host cell as described; (b) culturing the cultured host cell in a medium under conditions permitting expression of a polypeptide encoded by the vector and assembling of an antibody or fragment thereof, and (c) purifying the antibody or fragment thereof from the cultured cell or the medium of the cell.

### Methods of Production

Antibodies may be produced using recombinant methods and compositions, e.g., as described in U.S. Pat. No. 4,816,567. In one embodiment, an isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, a nucleic acid encoding an antibody, e.g., as described herein, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified, which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MVRC 5 cells; and FS4 cells. Other useful mammalian host cell lines include CHO cells, including DHFR-CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

b. IL-15 Polypeptides

In some embodiments, the IL-9 polypeptide comprises an IL-15 (e.g., human IL-15) or a variant/fragment thereof. In some embodiments, the IL-15 polypeptide comprises an amino acid sequence having at least 75K (e.g., 75%, 80%, 85%, 90%, 95%, 99%) sequence identity with an amino acid sequence selected from SEQ ID NOs: 89-92 or comprises an amino acid sequence of selected from SEQ ID NOs: 89-92.

TABLE 2
Representative Sequences of IL-15 Polypeptides

| SEQ ID | | OTHER |
|---|---|---|
| NO | SEQUENCES | INFORMATION |
| 89 | MNWIDVRYDLEKIESLIQSIHIDTTLYTDSDFHPSCKVTA | Recombinant |
| | MNCFLLELQVILHEYSNMTLNETVRNVLYLANSTLSSNK | mouse IL-15 |
| | NVAESGCKECEELEEKTFTEFLQSFIRIVQMFINTS | |
| 90 | NWVNVISDLKKIEDLIQSMHIDATLYTESDVHPSCKVTA | Homo sapiens |
| | MKCFLLELQVISLESGDASIHDTVENLIILANNSLSSNGNV | (Human) IL-15 |
| | TESGCKECEELEEKNIKEFLQSFVHIVQMFINTS | Processed |
| | | Mature Form |
| | | (49-162) |
| 91 | MKILKPYMRNTSILYYLCFLLNSHFLTEAGIHVFILGCVSV | Rattus |
| | GLPKTEANWIDVRYDLEKIESLIQFIHIDTTLYTDSDFHPS | norvegicus (Rat) |
| | CKVTAMNCFLLELQVILHEYSNMTLNETVRNVLYLANST | IL-15 |
| | LSSNKNVIESGCKECEELEERNFTEFLQSFIHIVQMFINTS | |
| 92 | MRISKPHLRSVSIQCYLCLLLNSHFLTEAGIHVFILGCFSA | Macaca mulatta |
| | GLPKTEANWVNVISDLKKIEDLIQSMHIDATLYTESDVHP | (Rhesus |
| | SCKVTAMKCFLLELQVISHESGDTDIHDTVENLIILANNIL | macaque) IL-15 |
| | SSNGNITESGCKECEELEEKNIKEFLQSFVHIVQMFINTS | |

The term “IL-15 polypeptide” and its abbreviation “IL-15” refer to a protein having the amino acid sequence of SEQ ID NO: 89, 90, 91, or 92, or a protein or polypeptide substantially homologous thereto. Such an IL-15 polypeptide has the biological properties of those of SEQ ID NO: 89, 90, or 91, including binding to an IL-15R (e.g., IL-15Rα) and stimulation of activation of effector NK cells and CD8+memory T cells. Both the naturally occurring human IL-15 polypeptide glycoprotein as well as recombinant human IL-15 polypeptide, heterodimeric IL-15 preparations consisting of IL-15 and IL-15Rα, and IL-15:IL-15Rα fusion proteins (e.g., IL-15 SA (Ahmad A, et al. Current HIV Research. 3 (3): 261-70), N/ALT-803 (Liu B, et al. The Journal of Biological Chemistry. 291 (46): 23869-23881), RLI-15 (Robinson T O, et al. Immunology Letters. 190: 159-168)), single chain recombinant human IL-15 (Vyas et al. Cell Culture and Tissue Engineering. 28(2) 2012)), hetIL-15/NIZ985 (Chertova E, et al. J Biol Chem. 2013 Jun. 21; 288(25):18093-103), NKTR-255 (Miyazaki T, et al. Journal for Immunotherapy of Cancer 2021; 9:e002024)).

The term “IL-15 polypeptide” or “IL-15” also covers chemically modified IL-15. Examples of chemically modified IL-15 polypeptides include those subjected to conformational change, addition or deletion of a sugar chain, and IL-15 polypeptide, to which a compound such as polyethylene glycol has been bound. Once purified and tested by standard methods or according to the method described in the examples below, IL-15 can be included in a pharmaceutical composition. Recombinant IL-15 polypeptide may be prepared via expression in eukaryotic cells, for example in CHO cells, or BHK cells, or HeLa cells by recombinant DNA technology or by endogenous gene activation.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. The term variant can be used to describe either polynucleotides or polypeptides.

As used herein, an IL-15 variant or fragment includes IL-15 fragments or segments that bind to an IL-15 receptor (e.g., IL-15Rα).

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide; for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also include polypeptides that are portions or subsequences of the parent polypeptide; for example, unique subsequences (e.g., as determined by standard sequence comparison and alignment techniques) of the polypeptides disclosed herein are also encompassed by the invention.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made.

In some embodiments, a variant of an IL-15 polypeptide may include one or more conservative modifications. The IL-15 polypeptide variant with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications. The IL-15 polypeptide with one or more conservative modifications may retain the desired functional properties, which can be tested using the functional assays known in the art.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

For determination of protein sequence identify, the values included are those defined as ‘Identities” by NCBI and do not account for residues that are not conserved but share similar properties (‘Po In some embodiments, the IL-15 polypeptide or a variant of an IL-15 polypeptide can be conjugated or linked to a detectable tag or a detectable marker (e.g., a radionuclide, a fluorescent dye, or an MRI-detectable label). In some embodiments, the detectable tag can be an affinity tag. The term “affinity tag,” as used herein, relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3×FLAG tag, a SUMO tag, and MBP-tag (MBP: maltose-binding protein). Further examples of affinity tags can be found in Kimple et al., Curr Protoc Protein Sci. 2013 Sep. 24; 73: Unit 9.9.

In some embodiments, the detectable tag can be conjugated or linked to the N- and/or C-terminus of an IL-15 polypeptide or a variant of an IL-15 polypeptide. The detectable tag and the affinity tag may also be separated by one or more amino acids. In some embodiments, the detectable tag can be conjugated or linked to the variant via a cleavable element. In the context of the present invention, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins that are joined by a peptide bond or via several peptides. The fusion protein may also comprise a peptide linker between the two domains.

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction etc., to provide a site for coupling. Methods for conjugation are well known by persons skilled in the art and are encompassed for use in the present invention. Linker moieties include, but are not limited to, chemical linker moieties, or for example, a peptide linker moiety (a linker sequence).

In some embodiments, the linker can be a peptide linker and a non-peptide linker. Examples of the peptide linker may include, without limitation, [S(G)n]m or [S(G)n]mS, where n may be an integer between 1 and 20, and m may be an integer between 1 and 10.

### B. Pharmaceutical Compositions and Administration

The present disclosure includes methods which comprise administering an anti-CD40 antibody or antigen binding fragment thereof and an IL-5 polypeptide to a subject wherein the antibodies are contained within a separate or combined (single) pharmaceutical composition.

The pharmaceutical compositions of this disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN), DNA conjugates, anhydrous absorption pastes, oil-in-water, and water-in-oil emulsions, emulsions carbowax (polyethylene glycols of various molecular weights), semi-solid gels, and semi-solid mixtures containing carbowax. See also Powell et al. PDA (1998) J Pharm Sci Technol 52:238-311.

Various delivery systems are known and can be used to administer the pharmaceutical composition of the present disclosure, e.g., encapsulation in liposomes, microparticles, microcapsules, recombinant cells capable of expressing the mutant viruses, receptor-mediated endocytosis (see, e.g., Wu et al., 1987, J. Biol. Chem. 262: 4429-4432). Methods of administration include, but are not limited to, intravesical, intradermal, intramuscular, intratumoral, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents.

A pharmaceutical composition comprising an anti-CD40 antibody or antigen binding fragment thereof and an IL-5 polypeptide can be delivered intratumorally, intravesically, subcutaneously, intraperitoneally, or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, e.g., Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous, and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Administration Regimens

The methods of the present disclosure may include administering to a subject an anti-CD40 antibody or antigen binding fragment thereof and/or an IL-5 polypeptide at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. The methods of the present disclosure may also include administering a single dose each of an anti-CD40 antibody or antigen binding fragment thereof and an IL-5 polypeptide.

In some embodiments, at least one of the anti-CD40 antibody or antigen binding fragment thereof and the IL-5 polypeptide is administered to the patient once a day, once every two days, once every three days, once every four days, once every five days, once every week, once every two weeks, or once every three weeks.

In some embodiments, the anti-CD40 antibody or antigen binding fragment thereof and the IL-5 polypeptide are administered concurrently to the patient.

In some embodiments, the methods may include sequentially administering to the subject the anti-CD40 antibody or antigen binding fragment thereof and the IL-5 polypeptide. In some embodiments, the anti-CD40 antibody or antigen binding fragment thereof is administered to the patient before or after the IL-5 polypeptide.

As used herein, “sequentially administering” means that each dose of the anti-CD40 antibody or antigen binding fragment thereof and the IL-15 polypeptide is administered to the subject at a different point in time, e.g., on different days separated by a predetermined interval (e.g., hours, days, weeks or months). The present disclosure includes methods which comprise sequentially administering to the patient a single initial dose of the anti-CD40 antibody or antigen binding fragment thereof or the IL-15 polypeptide, followed by one or more secondary doses of the anti-CD40 antibody or antigen binding fragment thereof or the IL-15 polypeptide, and optionally followed by one or more tertiary doses of the anti-CD40 antibody or antigen binding fragment thereof or the IL-15 polypeptide.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the anti-CD40 antibody or antigen binding fragment thereof or the IL-15 polypeptide. In some embodiments, however, the amount contained in the initial, secondary, and/or tertiary doses varies from one another (e.g., adjusted up or down as appropriate) during the course of treatment. In some embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, a dose of the anti-CD40 antibody or antigen binding fragment thereof or the IL-15 polypeptide, which is administered to a patient prior to the administration of the very next dose in the sequence with no intervening doses.

In some embodiments, the methods may include administering to a patient any number of secondary and/or tertiary doses of the anti-CD40 antibody or antigen binding fragment thereof or the IL-15 polypeptide. For example, in some embodiments, only a single secondary dose is administered to the patient. In other embodiments, two or more (e.g., 2, 3, 4, 5, 6, 7, 8, or more) secondary doses are administered to the patient. Likewise, in some embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, one or more doses of the anti-CD40 antibody or antigen binding fragment thereof or the IL-15 polypeptide are administered at the beginning of a treatment regimen as “induction doses” on a more frequent basis (twice a week, once a week, or once in 2 weeks) followed by subsequent doses (“consolidation doses” or “maintenance doses”) that are administered on a less frequent basis (e.g., once in 4-12 weeks).

### Dosage

The amount of the anti-CD40 antibody or antigen binding fragment thereof or the IL-15 polypeptide administered to a subject according to the methods disclosed herein is, generally, a therapeutically effective amount. As used herein, the term “therapeutically effective amount” means an amount of the anti-CD40 antibody or antigen binding fragment thereof and/or the IL-15 polypeptide that results in one or more of: (a) a reduction in the severity or duration of a symptom or an indication of cancer, e.g., a tumor lesion; (b) inhibition of tumor growth, or an increase in tumor necrosis, tumor shrinkage and/or tumor disappearance; (c) delay in tumor growth and development; (d) inhibition of tumor metastasis; (e) prevention of recurrence of tumor growth; (f) increase in survival of a subject with a cancer; and/or (g) a reduction in the use or need for conventional anti-cancer therapy (e.g., elimination of need for surgery or reduced or eliminated use of chemotherapeutic or cytotoxic agents) as compared to an untreated subject, a subject treated with monotherapy, or a subject treated with the therapeutic agents disclosed herein (the anti-CD40 antibody or antigen binding fragment thereof and the IL-15 polypeptide).

In some embodiments, a therapeutically effective amount of the anti-CD40 antibody or antigen binding fragment thereof or the IL-15 polypeptide can be from about 0.05 mg to about 1500 mg, from about 1 mg to about 800 mg, from about 5 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the anti-CD40 antibody or antigen binding fragment thereof or the IL-15 polypeptide is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, or about 1500 mg.

The amount of an anti-CD40 antibody or antigen binding fragment thereof or an IL-15 polypeptide contained within an individual dose may be expressed in terms of milligrams of antibody per kilogram of subject body weight (i.e., mg/kg). In some embodiments, the anti-CD40 antibody or antigen binding fragment thereof used in the methods disclosed herein may be administered to a subject at a dose of about 0.0001 to about 100 mg/kg of subject body weight. In some embodiments, an anti-CD40 antibody or antigen binding fragment thereof or an IL-15 polypeptide may be administered at a dose of about 0.1 mg/kg to about 20 mg/kg of a patient's body weight. In some embodiments, the methods of the present disclosure comprise administration of an anti-CD40 antibody or antigen binding fragment thereof or an IL-15 polypeptide at a dose of about 1 mg/kg to 3 mg/kg, 1 mg/kg to 5 mg/kg, 1 mg/kg to 10 mg/kg, 1 mg/kg, 3 mg/kg, 5 mg/kg, or 10 mg/kg of a patient's body weight.

In some embodiments, each dose comprises 0.1-10 mg/kg (e.g., 0.3 mg/kg, 1 mg/kg, 3 mg/kg, or 10 mg/kg) of the subject's body weight. In certain other embodiments, each dose comprises 5-1500 mg of the anti-CD40 antibody or antigen binding fragment thereof or the IL-15 polypeptide, e.g., 5 mg, 10 mg, 15 mg, 20 mg, 25 mg, 30 mg, 40 mg, 45 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1550 mg, 1300 mg, 1350 mg, 1400 mg, 1450 mg, or 1500 mg of the anti-CD40 antibody or antigen binding fragment thereof or the IL-15 polypeptide.

In some embodiments, the methods of the present disclosure further include administering to a subject an additional therapeutic agent or therapy. The additional therapeutic agent or therapy may be administered for increasing anti-tumor efficacy, for reducing toxic effects of one or more therapies and/or for reducing the dosage of one or more therapies. In various embodiments, the additional therapeutic agent or therapy may include one or more of: radiation, surgery, a cancer vaccine, imiquimod, an anti-viral agent (e.g., cidofovir), photodynamic therapy, a PD-1/PD-L1 pathway inhibitor (e.g., an anti-PD-1 antibody, an anti-PD-L1 antibody), a lymphocyte activation gene 3 (LAG3) inhibitor (e.g., an anti-LAG3 antibody, a glucocorticoid-induced tumor necrosis factor receptor (GITR) agonist (e.g., an anti-GITR antibody), a T-cell immunoglobulin and mucin containing −3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an indoleamine-2,3-dioxygenase (IDO) inhibitor, a CD28 activator, a vascular endothelial growth factor (VEGF) antagonist (e.g., a “VEGF-Trap” such as aflibercept, or an anti-VEGF antibody or antigen-binding fragment thereof (e.g., bevacizumab, or ranibizumab) or a small molecule kinase inhibitor of VEGF receptor (e.g., sunitinib, sorafenib, or pazopanib)), an angiopoietin-2 (Ang2) inhibitor, a transforming growth factor beta (TGFβ) inhibitor, an epidermal growth factor receptor (EGFR) inhibitor, an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a Bacillus Calmette-Guerin (BCG) therapy, a vaccine (e.g., Bacillus Calmette-Guerin (BCG)), granulocyte-macrophage colony-stimulating factor (GM-CSF), a second oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), an IL-6R inhibitor, an IL-4R inhibitor, an IL-10 inhibitor, a cytokine such as IL-2, IL-7, IL-12, and IL-21, an antibody drug conjugate, an anti-inflammatory drug such as a corticosteroid, a non-steroidal anti-inflammatory drug (NSAID), cryotherapy, anti-HPV therapy, laser therapy, electrosurgical excision of cells with HPV, and combinations thereof.

In some embodiments, the methods further comprise administering an additional therapeutic agent, such as an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mitotane (O, P′-(DDD)), biologics (e.g., antibodies and interferons) and radioactive agents. As used herein, “a cytotoxin or cytotoxic agent” also refers to a chemotherapeutic agent and means any agent that is detrimental to cells. Examples include TAXOL (paclitaxel), temozolomide, cytochalasin B, gramicidin D, ethidium bromide, emetine, cisplatin, mitomycin, etoposide, teniposide, vincristine, vinblastine, colchicine, doxorubicin, daunorubicin, dihydroxy anthracene dione, mitoxantrone, mithramycin, actinomycin D, 1-dihydrotestosterone, glucocorticoids, procaine, tetracaine, lidocaine, propranolol, and puromycin and analogs or homologs thereof.

The present disclosure also provides kits comprising an anti-CD40 antibody or antigen binding fragment thereof and an IL-15 polypeptide, in combination with written instructions for use of a therapeutically effective amount of a combination of the anti-CD40 antibody or antigen binding fragment thereof and the IL-15 polypeptide for treating or inhibiting the growth of a tumor of a patient.

In another aspect, this disclosure provides a kit comprising a pharmaceutically acceptable dose unit of the anti-CD40 antibody or antigen-binding fragment thereof and/or the IL-15 polypeptide or the pharmaceutical composition as described herein.

In some embodiments, the kit also includes a container that contains the composition and optionally informational material. The informational material can be descriptive, instructional, marketing or other material that relates to the methods described herein and/or the use of the agents for therapeutic benefit. In an embodiment, the kit also includes an additional therapeutic agent, as described herein. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

The kit can include one or more containers for the composition. In some embodiments, the kit contains separate containers, dividers, or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading. Such a kit may optionally contain a syringe to allow for injection of the antibody contained within the kit into an animal, such as a human.

### C. Additional Definitions

To aid in understanding the detailed description of the compositions and methods according to the disclosure, a few express definitions are provided to facilitate an unambiguous disclosure of the various aspects of this disclosure. Unless otherwise defined, all technical and scientific terms used herein have the same meaning as commonly understood by one of ordinary skill in the art to which this disclosure belongs.

CD40 refers to “TNF receptor superfamily member 5” (TNFRSF5). Unless otherwise indicated, or clear from the context, references to CD40 herein refer to human CD40 (“huCD40”), and anti-CD40 antibodies refer to anti-human CD40 antibodies. Human CD40 is further described at GENE ID NO: 958 and MIM (Mendelian Inheritance in Man): 109535. The sequence of human CD40 can be accessed by the Accession Code NP_001241.1.

CD40 interacts with CD40 ligand (CD40L), which is also referred to as TNFSF5, gp39, and CD154. Unless otherwise indicated, or clear from the context, references to CD40L herein refer to human CD40L (“huCD40L”). Human CD40L is further described at GENE ID NO: 959 and MIM: 300386. The sequence of human CD40L can be accessed by the Accession Code NP_000065.1.

This disclosure encompasses isolated or substantially purified nucleic acids, peptides, polypeptides or proteins. In the context of the present disclosure, an “isolated” nucleic acid, DNA or RNA molecule or an “isolated” polypeptide is a nucleic acid, DNA molecule, RNA molecule, or polypeptide that exists apart from its native environment and is therefore not a product of nature. An isolated nucleic acid, DNA molecule, RNA molecule or polypeptide may exist in a purified form or may exist in a non-native environment such as, for example, a transgenic host cell. A “purified” nucleic acid molecule, peptide, polypeptide or protein, or a fragment thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein, peptide or polypeptide that is substantially free of cellular material includes preparations of protein, peptide or polypeptide having less than about 30%, 20%, 10%, or 5% (by dry weight) of contaminating protein. When the protein or biologically active portion thereof, is recombinantly produced, preferably culture medium represents less than about 30%, 20%, 10%, or 5% (by dry weight) of chemical precursors or non-protein-of-interest chemicals.

The terms polypeptide, peptide, and protein are used interchangeably herein.

The term “recombinant,” as used herein, refers to antibodies or antigen-binding fragments thereof or IL-15 polypeptides of this disclosure created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of this disclosure to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

The term “Kassoc” or “Ka,” as used herein, is intended to refer to the association rate of a particular antibody-antigen interaction, whereas the term “Kdis” or “Kd,” as used herein, is intended to refer to the dissociation rate of a particular antibody-antigen interaction. The term “KD,” as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface plasmon resonance, preferably using a biosensor system such as a BIACORE system.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In some embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance). Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “epitope” as used herein refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In some embodiments, epitopes may include determinants that are chemically active surface groupings of molecules such as amino acids, sugar side chains, phosphoryl groups, or sulfonyl groups, and, In some embodiments, may have specific three-dimensional structural characteristics, and/or specific charge characteristics. An epitope typically includes at least 3, 4, 5, 6, 7, 8, 9, 10, 11, 12, 13, 14, or 15 amino acids in a unique spatial conformation. Methods for determining what epitopes are bound by a given antibody (i.e., epitope mapping) are well known in the art and include, for example, immunoblotting and immune-precipitation assays, wherein overlapping or contiguous peptides from a Spike or S protein are tested for reactivity with a given antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “epitope mapping” refers to the process of identification of the molecular determinants for antibody-antigen recognition.

The term “binds to an epitope” or “recognizes an epitope” with reference to an antibody or antibody fragment refers to continuous or discontinuous segments of amino acids within an antigen. Those of skill in the art understand that the terms do not necessarily mean that the antibody or antibody fragment is in direct contact with every amino acid within an epitope sequence.

The term “binds to the same epitope” with reference to two or more antibodies means that the antibodies bind to the same, overlapping or encompassing continuous or discontinuous segments of amino acids. Those of skill in the art understand that the phrase “binds to the same epitope” does not necessarily mean that the antibodies bind to or contact exactly the same amino acids. The precise amino acids that the antibodies contact can differ. For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., neuronal disorders, autoimmune diseases, and cardiovascular diseases), and/or who has been diagnosed with inflammatory disorders. In some embodiments, the subject is a mammal. In some embodiments, the subject is human.

As used herein, the term “disease” is intended to be generally synonymous and is used interchangeably with, the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

As used herein, the term “treating” or “treatment” of any disease or disorder refers in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically (e.g., stabilization of a discernible symptom), physiologically (e.g., stabilization of a physical parameter), or both. In yet another embodiment, “treating” or “treatment” refers to preventing or delaying the onset or development or progression of the disease or disorder.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “therapeutic effect” is art-recognized and refers to a local or systemic effect in animals, particularly mammals, and more particularly humans caused by a pharmacologically active substance.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, the term “composition” or “pharmaceutical composition” refers to a mixture of at least one component useful within the disclosure with other components, such as carriers, stabilizers, diluents, dispersing agents, suspending agents, thickening agents, and/or excipients. The pharmaceutical composition facilitates administration of one or more components of this disclosure to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of this disclosure, and are physiologically acceptable to the subject. Supplementary active compounds may also be incorporated into the compositions.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, “administering” refers to the physical introduction of a composition comprising a therapeutic agent to a subject, using any of the various methods and delivery systems known to those skilled in the art. Example routes of administration for antibodies described herein include intravenous, intraperitoneal, intramuscular, subcutaneous, intratumoral, intravesical, spinal or other parenteral routes of administration, for example, by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of this disclosure.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 4%1, 3%1, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percent, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither, or both limits are included in the smaller ranges is also encompassed within the disclosure, subject to any specifically excluded limit in the stated range. Where the stated range includes one or both of the limits, ranges excluding either or both of those included limits are also included in the disclosure.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of this disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of this disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. Examples

### Combination of IL-15 and Intravesical Anti-hCD40 Antibodies for Bladder Cancer Therapy

Antibody-based therapeutics have become one of the fastest growing classes of anti-cancer agents, starting with the introduction of tumor-targeting antibodies against cell surface antigens (e.g., CD20 on malignant B cells) and more recently as agents targeting key regulatory immune pathways (e.g., PD-1/L1, CTLA-4, CD40). Antibodies have two ends, one end with bivalent Fabs recognizing the target of interest and the other end with a single Fc, which engages a diversity of immune cells and plays a critical role in determining the final effector function of a given antibody. Importantly, optimal in vivo activity requires careful consideration of two key structural characteristics of the Fc, the subclass (e.g., IgG1 vs. IgG4) or composition of the conserved biantennary N-linked glycan (e.g., afucosylation to enhance binding to FcγRIIIA).

For antibodies targeting immune regulatory pathways, we have defined the role of the Fc region for antibodies targeting the inhibitory immune checkpoints PD-1 and CTLA-4, as well as more recently described the critical aspects of the antibody Fc governing the activity of agonistic antibodies targeting CD40 on antigen-presenting cells. Importantly, as opposed to the requirement for binding activating FcγRs for tumor-targeting antibodies, agonist anti-hCD40 antibodies require engagement of the inhibitory FcγRIIB, which facilitates receptor crosslinking and proper oligomerization of this trimeric TNF-superfamily receptor needed for optimal CD40 signaling. Using multiple humanized in vivo orthotopic bladder tumor models, It was also recently described that Fc-optimized agonist anti-hCD40 antibodies administered locally via an intravesical route can provide potent, durable, and systemic anti-tumor control with a favorable systemic toxicity profile (Garris, C., et al. Science Translational Medicine in press (2021)). This approach is now entering clinical phase testing.

While the studies have been pivotal to the understanding of how antibodies can be optimized as single-agent therapies, a combination therapy with a specific focus on the IL-15 pathway was investigated. IL-15 is a 14-15 kDa member of the 4-alpha-helix bundle family of cytokines acting through a heterotrimeric receptor involving IL-2/IL-15Rβ (CD122, subunits shared with IL-2), the common gamma chain (γc) (CD132, shared with IL-2, IL-4, IL-7, IL-9, IL-21), and the IL-15-specific receptor subunit IL-15Rα (CD215). IL-15 functions as a cell-surface molecule that acts as part of an immunological synapse with IL-15 and IL-15Rα produced on adjacent DCs, monocytes, and other myeloid cells in response to inflammatory stimuli, such as interferon and/or Toll-like receptor ligands. IL-15Rα presents IL-15 in trans to NK and CD8 T cells expressing IL-2/IL-15Rβ and γc, and has been shown in many model systems to be a potent stimulator of T and NK cell functions. It was demonstrated (in mice) that systemic IL-15 therapy leads to enhanced expression of activating FcγRs and anti-tumor activity when given with anti-tumor antibodies. In addition, in contrast to IL-2, IL-15 does not meaningfully activate Tregs and participates less than IL-2 in the capillary leak syndrome. However, IL-15 has not been investigated with the use of Fc-optimized anti-human CD40 agonist antibodies, employing humanized orthotopic tumor models, via a local (e.g., intravesical) therapeutic route, or in the context of certain cancers, such as bladder cancer.

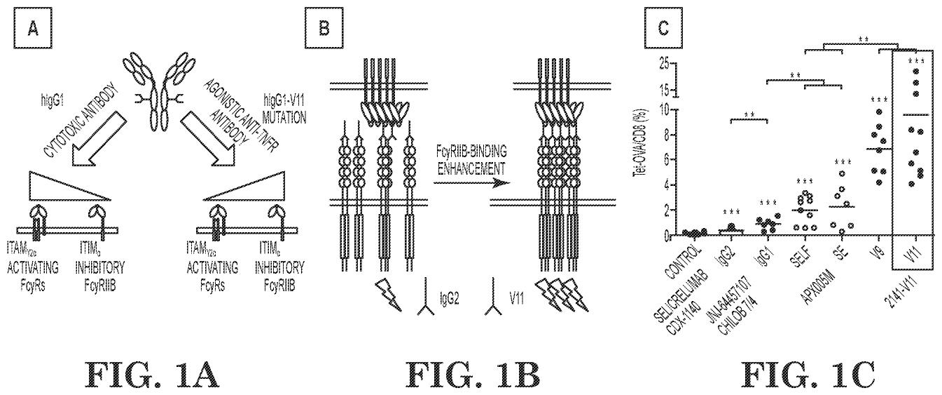

It has been a focus to generate antibodies aimed at stimulating antigen-presenting cells (APCs, e.g., dendritic cells (DCs), B cells, and macrophages) within the tumor microenvironment. Specifically, as noted above, it was found that antibodies against CD40 on the surface of APCs are potent activators of the immune response against infection and cancer. For optimal in vivo activity, these agonistic antibodies require crosslinking facilitated by binding of the Fc to the inhibitory Fc receptor (FcγRIIB). Using this information, an enhanced anti-hCD40 agonist antibody (2141-V11) was engineered, which carries 5 point mutations in the Fc domain to selectively increase binding to hFcγRIIB (FIG. 1). In models humanized for CD40 and human FcγRs (hCD40/hFcγR), 2141-V11 significantly enhanced immune stimulatory activities in vivo, which translated to superior anti-tumor activity across several tumor types. However, as has been shown with other CD40 agonist antibodies, the 2141-V11 therapeutic potential is limited by its systemic toxicity by inducing thrombocytopenia and transaminitis. To overcome this clinically significant hurdle, it was demonstrated that local (intratumoral or intravesical) administration of 2141-V11 stimulates a potent and durable immune response both locally and systemically. Importantly, these studies also further validate the importance of using species-matched IgG Fc-FcγR systems to reliably model antibody activity and toxicity in vivo. Supported by this preclinical data, locally-administered 2141-V11 antibody is now being investigated clinically in patients across several different disease types, including cutaneous tumors (NCT04059588), glioblastoma (NCT04547777), and bladder cancer.

While these data support a role for 2141-V11 in expanding antigen specific T cells to mediate anti-tumor activity at both local and distant sites of disease, therapeutic combinations with 2141-V11 were further investigated to optimize tumor responses in vivo. In addition to antigen stimulation, T cells also require supportive signals from cytokines to persist in vivo. One of the most potent has been the gamma chain (γc) cytokine family member interleukin 15 (IL-15). However, as a monotherapy, IL-15 treatment results in paralysis of CD4 T cells mediated through SOCS3/STAT5B signaling. This paralysis of CD4 T cell “helper” activity inhibits the generation of tumor-specific CD8 T cells, an effect that can be successfully rescued by the addition of CD40 agonists.

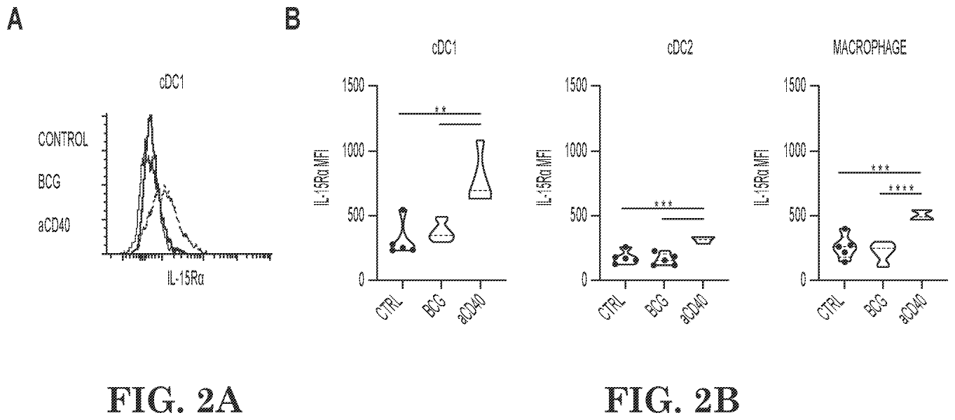

Using humanized hCD40/hFcγR mice bearing aggressive orthotopic MB49 bladder tumors, it was demonstrated that local intravesical treatment with anti-human CD40 agonist antibodies, including the Fc-optimized 2141-V11 antibody, leads to upregulation of IL-15Rα on DCs and other myeloid cells within the bladder tumor microenvironment (FIGS. 2A and 2B). This IL-15Rα upregulation is not similarly observed upon treatment with BCG, a live-attenuated bacterium routinely used clinically as a therapeutic immune stimulus in the treatment of localized bladder cancer, supporting IL-15 as a combinatorial target that may uniquely synergize with CD40-directed therapy.

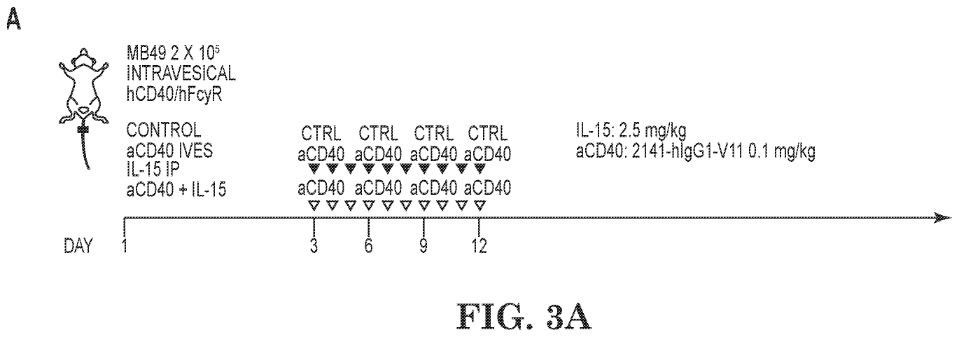

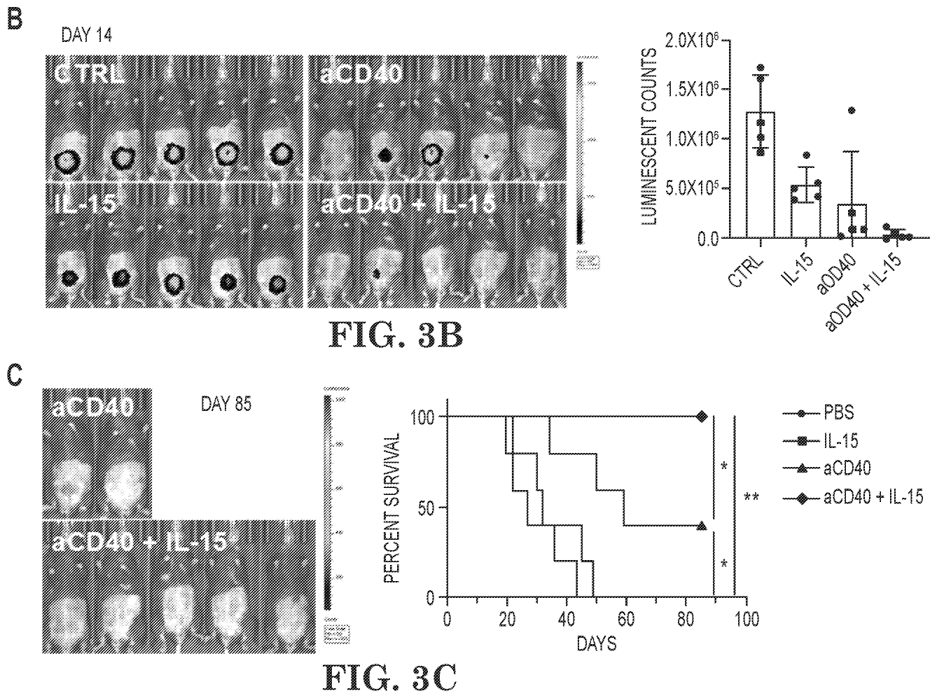

It was further demonstrated that the combination of intravesically-administered 2141-V11 antibody with systemically-administered IL-15 (FIG. 3A) results in significantly decreased tumor burden (FIG. 3B, as measured by tumor cell bioluminescence), as well as notably improved rates of complete response and overall survival (FIG. 3C).

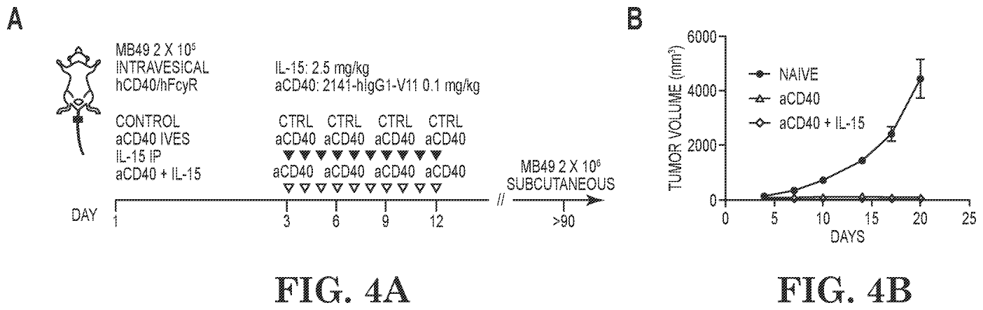

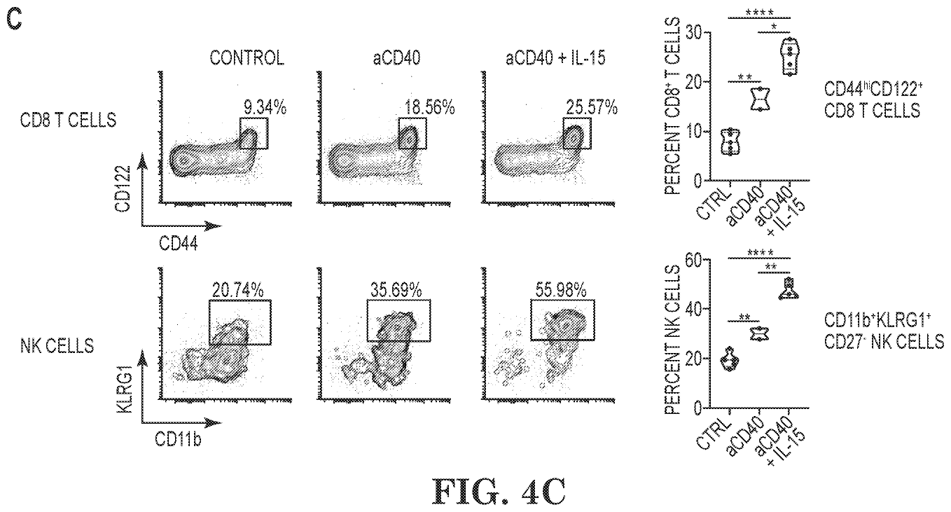

The ability of combined treatment with 2141-V11 and IL-15 to induce long-term protective systemic immunity was investigated. Mice surviving long-term (>90 days) from the initial therapy were rechallenged at a ten-fold higher dose of tumor cells in a different tissue compartment (subcutaneous rather than within the bladder) in the absence of any additional therapy (FIG. 4A). Mice previously treated with 2141-V11 and IL-15 rejected tumor rechallenge (FIG. 4B), indicating that this treatment is capable of inducing an in situ vaccination effect driving durable protective systemic anti-tumor immunity. This was also associated with a notable enhancement in the proportions of circulating CD44^{hi}CD122⁺ CD8 T cells, which have been described as an important memory-phenotype T cell population involved in the anti-tumor memory response, as well as CD11b⁺KLRG1⁺CD27⁻ NK cells, a mature phenotype likewise implicated in durable anti-tumor immunity (FIG. 4C).

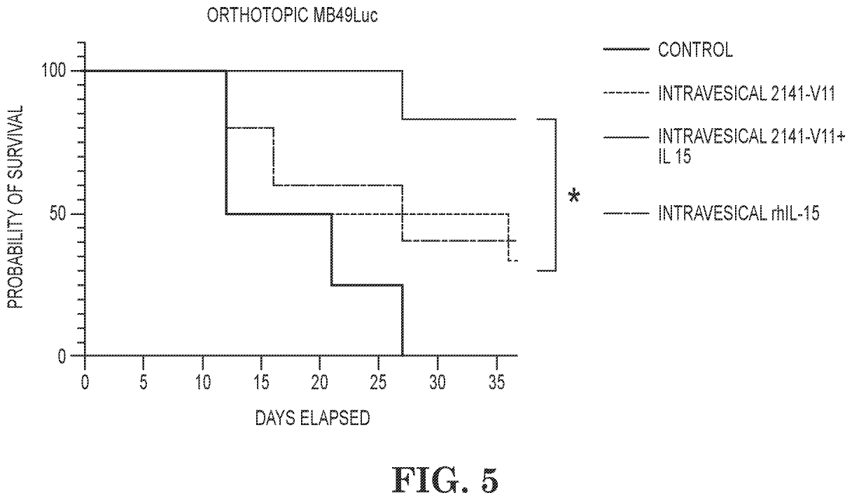

The ability of combined treatment with 2141-V11 and recombinant human IL-15 given intravesically to the bladder to enhance antitumor activity was investigated (FIG. 5). Combination therapy with the Fc-optimized anti-CD40 agonist antibody 2141-V11 and human recombinant IL-15 enhances primary anti-tumor activity. Humanized hCD40/hFcyR mice bearing orthotopic MB49 bladder tumors were treated with intravesical 2141-V11 and/or intravesical recombinant human IL-15 or control (isotype-matched control antibody and/or vehicle) and followed for survival.

In total, these data support this combination therapy as a robust inducer of both primary and memory anti-tumor responses.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for treating a cancer, comprising administering to a subject in need thereof a therapeutically effective amount of an interleukin-15 (IL-15) polypeptide in combination with a therapeutically effective amount of an isolated antibody or antigen binding fragment thereof that specifically binds to human CD40, wherein the anti-CD40 agonist antibody is selected from 2141, CP-870,893, ChiLob, 12D6, 5F11, 8E8, 5G7, 19G3, APX005M, ADC-1013, CDX-1140, SEA-CD40, SGN-CD40, ABBV-927, and Fc variants thereof.

2. The method of claim 1, wherein the anti-CD40 agonist antibody is selected from 2141-V11, CP-870,893-V11, ChiLob-V11, 12D6-V11, 5F11-V11, 8E8-V11, 5G7-V11, 19G3-V11, APX005M-V11, ADC-1013-V11, CDX-1140-V11, SEA-CD40-V11, SGN-40-V11, and ABBV-927-V11.

3. The method of any one of the preceding claims, wherein the anti-CD40 antibody or antigen binding fragment thereof is administered intratumorally.

4. The method of any one of the preceding claims, wherein the cancer is bladder cancer.

5. The method of claim 4, wherein the anti-CD40 antibody or antigen binding fragment thereof is administered intravesically.

6. The method of any one of the preceding claims, wherein one or more doses of the IL-15 polypeptide are administered to the subject prior to or after administering to the subject one or more doses of the anti-CD40 antibody or antigen binding fragment thereof.

7. The method of any one of claims 1-4, wherein one or more doses of the IL-15 polypeptide are administered to the subject concomitantly with administering to the subject one or more doses of the anti-CD40 antibody or antigen binding fragment thereof.

8. The method of any one of the preceding claims, wherein the IL-15 polypeptide is administered intravenously, subcutaneously, intraperitoneally, intratumorally, or intravesically.

9. The method of any one of the preceding claims, wherein the therapeutically effective amount of the anti-CD40 antibody or antigen binding fragment thereof comprises from 0.1 to 20 mg/kg of the subject's body weight.

10. The method of any one of the preceding claims, wherein the therapeutically effective amount of the IL-15 polypeptide comprises from 0.1 to 20 mg/kg of the subject's body weight.

11. The method any one of the preceding claims, wherein the anti-CD40 antibody or antigen binding fragment thereof comprises three heavy chain complementarity determining regions (HCDR1, HCDR2, and HCDR3) of a heavy chain having an amino acid sequence selected from the group consisting of SEQ ID NOs:79, 81-88 and three light chain complementarity determining regions (LCDR1, LCDR2, and LCDR3) of a light chain having the amino acid sequence of SEQ ID NO: 80.

12. The method any one of the preceding claims, wherein the anti-CD40 antibody or antigen binding fragment thereof comprises a heavy chain variable region (HCVR) a heavy chain having an amino acid sequence selected from the group consisting of SEQ ID NOs:79, 81-88 and a light chain variable region (LCVR) of a light chain having the amino acid sequence of SEQ ID NO: 80.

13. The method any one of the preceding claims, wherein the anti-CD40 antibody or antigen binding fragment thereof comprises an Fc region modified to enhance specificity of binding to FcγRIIb, wherein the Fc region comprises a sequence selected from the group consisting of SE (SEQ ID NO: 66), SELF (SEQ ID NO: 67), P238D (SEQ ID NO: 68), V4 (SEQ ID NO: 69), V4 D270E (SEQ ID NO: 70), V7 (SEQ ID NO: 71), V8 (SEQ ID NO: 72), V9 (SEQ ID NO: 73), V9 D270E (SEQ ID NO: 74), V11 (SEQ ID NO: 75), and V12 (SEQ ID NO: 76).

14. The method any one of the preceding claims, wherein the anti-CD40 antibody or antigen binding fragment thereof comprises a heavy chain comprising the amino acid sequence selected from the group consisting of SEQ ID NO: 79, 81-88 and a light chain comprising the amino acid sequence of SEQ ID NO: 80.

15. The methods of any one of the preceding claims, wherein the IL-15 polypeptide comprises an IL-15 or a variant thereof.

16. The methods of any one of the preceding claims, wherein the IL-15 polypeptide comprises an amino acid sequence having at least 80% sequence identity with an amino acid sequence selected from SEQ ID NOs: 89-92 or comprises an amino acid sequence selected from SEQ ID NOs: 89-92.

17. The method of any one of the preceding claims, further comprising administering the subject an additional therapeutic agent or therapy, wherein the additional therapeutic agent or therapy is selected from radiation, surgery, a chemotherapeutic agent, a cancer vaccine, a PD-1 inhibitor, a PD-L1 inhibitor, a B7-H3 inhibitor, a B7-H4 inhibitor, a lymphocyte activation gene 3 (LAG3) inhibitor, a T cell immunoglobulin and mucin-domain containing-3 (TIM3) inhibitor, a galectin 9 (GAL9) inhibitor, a V-domain immunoglobulin (Ig)-containing suppressor of T-cell activation (VISTA) inhibitor, a Killer-Cell Immunoglobulin-Like Receptor (KIR) inhibitor, a B and T lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, a Bacillus Calmette-Guerin (BCG) therapy, a Bacillus Calmette-Guerin (BCG) vaccine, granulocyte-macrophage colony-stimulating factor (GM-CSF), a cytotoxin, an interleukin 6 receptor (IL-6R) inhibitor, an interleukin 4 receptor (IL-4R) inhibitor, an IL-10 inhibitor, IL-2, IL-7, IL-12, IL-21, an antibody-drug conjugate, an anti-inflammatory drug, and combinations thereof.

18. The method of any one of claims 1-3 and 6-17, wherein the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

19. The method of any one of the preceding claims, wherein the treatment produces a therapeutic effect selected from one or more of: delay in tumor growth, reduction in tumor cell number, tumor regression, prevention or delay of tumor recurrence, increase in survival, partial response, and complete response.

20. The method of claim 19, wherein the tumor growth is inhibited by at least 50% as compared to an untreated patient.

21. The method of any one of the preceding claims, wherein the tumor growth is inhibited by at least 50% as compared to a patient administered the anti-CD40 antibody or antigen binding fragment thereof or the IL-15 polypeptide as a monotherapy.

22. A combination or a kit comprising an anti-CD40 antibody or antigen binding fragment thereof or an IL-15 polypeptide for use in a method of treating a cancer, the method comprising:
selecting a patient with a cancer; and
administering to the patient a therapeutically effective amount of anti-CD40 antibody or antigen binding fragment thereof in combination with a therapeutically effective amount of the IL-15 polypeptide,
wherein the anti-CD40 antibody is is selected from 2141-V11, CP-870,893-V11, ChiLob-V11, 12D6-V11, 5F11-V11, 8E8-V11, 5G7-V11, 19G3-V11, APX005M-V11, ADC-1013-V11, CDX-1140-V11, SEA-CD40-V11, SGN-40-V11, and ABBV-927-V11.

23. The combination or kit of claim 22, wherein the 2141-V11 comprises a heavy chain having the amino acid sequence selected from the group consisting of SEQ ID NO: 79, 81-88 and a light chain having the amino acid sequence of SEQ ID NO: 80.
